(19) United States
(12) Patent Application Publication
(10) Pub. No.: US 2026/0112447 A1
(43) Pub. Date: Apr. 23, 2026

(54) Protein engineering using a multimodal and retrieval augmented generative model of protein families

(71) Applicant: NE47 Bio, Inc., Hillsborough, NC (US)

(72) Inventors: Tristan Bepler, Hillsborough, NC (US); Timothy F. Truong, JR., Flushing, NY (US)

(21) Appl. No.: 19/415,400

(22) Filed: Dec. 10, 2025

Related U.S. Application Data
(63) Continuation-in-part of application No. 18/443,534, filed on Feb. 16, 2024.
(60) Provisional application No. 63/730,045, filed on Dec. 10, 2024.
(60) Provisional application No. 63/756,477, filed on Feb. 10, 2025.
(60) Provisional application No. 63/846,320, filed on Jul. 18, 2025.

(51) Int. Cl. G16B 15/20; G16B 15/30; G16B 40/20

(52) U.S. Cl. CPC G16B 15/20; G16B 15/30; G16B 40/20

(57) ABSTRACT

A multimodal generative model of protein families is provided to jointly model the sequences and structures of proteins within a protein family. The model is used for controllable protein generation and representation learning. The model leverages in-context learning to infer underlying evolutionary constraints that give rise to the family's characteristic sequence features, structural architectures, and/or functional properties. These inferred evolutionary constraints, coupled with a flexible grammar for specifying explicit sequence and structural constraints, enables the controlled generation of novel family members, including variants with optimized or enhanced characteristics relevant to their function. The model is as an encoder-decoder transformer, preferably one encoder, and two decoders. The encoder processes a user-provided prompt comprising a set of proteins and that guides the two decoders toward generating novel proteins with desired characteristics.

### BACKGROUND

Proteins carry out most of the biological functions at the molecular level of life. The function of a protein is encoded by its specific amino acid sequence and the three-dimensional structure that the sequence folds into. Engineering proteins for novel and enhanced function is a key problem in pharmaceuticals and biotechnology and involves designing novel sequences or modifying existing natural proteins for these purposes. Deep mutational scans and directed evolution experiments have been used to successfully design novel proteins, but they can be costly and difficult to implement, which makes these experimental methods inapplicable for many proteins and functions of interest. Accurate computational models of sequence-function relationships can narrow down the protein sequence search space, reduce the need for expensive experiments, and enable the design of more novel proteins and functions.

Protein language models have emerged as promising methods for understanding and designing protein sequences. In particular, generative models offer a natural way to produce new protein designs. By training on large corpuses of natural protein sequences, these models learn evolutionary constraints on sequence space. They can then be used either to generate realistic sequences directly by sampling, or to identify promising protein sequence variants by predicting the relative fitness of the variants of interest using the sequence likelihoods as a proxy.

Traditionally, family-specific models learn evolutionary constraints specific to the protein family of interest by training on a multiple sequence alignment (MSA) of homologous sequences. However, this is ineffective for protein families with few sequences due to the lack of sufficient training data and inability to exploit information across families. These models also assume that MSAs are accurate, and they cannot model novel insertions or deletions (indels) not present in the training MSA.

Evolutionary sequence models are well established methods in biological sequences analysis. To model protein families, these models search large protein sequence databases for homologs, align the positions of these homologs in an MSA, and then fit statistical sequence models to the MSA. Common models include site independent models, profile HMMs, and coupling models. Newer variants incorporate higher order correlations between sequence positions by training a VAE or by building phylogenetic trees. These approaches are often referred to as “alignment-based” and must be fit on a family-by-family basis, requiring large numbers of members to generalize. A significant limitation of these models is that they assume the MSA is an accurate model of the evolutionary process generating the sequences, when in fact, MSA algorithms inevitably make alignment errors; regions with long insertions or lots of gaps can be particularly problematic.

Unconditional protein language models that do not condition on homologs at inference have emerged as powerful methods for understanding and generating protein sequences. Both bidirectional models and autoregressive generative models have demonstrated competitive performance for variant function prediction. The latter type of model has the advantage of being able to score indels, but both cannot integrate evolutionary context not present in the trained model parameters. In contrast to family-specific evolutionary sequence models trained on sequences derived from a specific protein family, these protein language models are pre-trained on large protein databases that span all known sequences. This enables them to learn evolutionary constraints that generalize across families to improve predictions for small families with few homologs, but they generally underperform family-specific models for larger families.

Hybrid models such as Tranception and TranceptEVE combine unconditional language models with family-specific models to enable specialization to protein families. Nonetheless, it is unclear how to use these models to generate sequences with novel indels, and predictions from the family-specific models do not directly benefit from transfer learning across families.

Conditional protein language models fit between the unconditional and family-specific paradigms. Only a few works have explored this direction to date. Masked language models of whole MSAs are able to integrate evolutionary context directly for conditioning site predictions, but they are unable to model insertions in the alignment. Ram and Bepler use an encoder-decoder framework to generate new sequences conditioned on an MSA, which removes the insertion limitation of Rao et al., but still requires conditioning on aligned input sequences. Notin et al. combine predictions from an unconditional language model and an alignment-based model and show that integrating retrieval-based methods with protein language models can improve variant function prediction performance. However, the reliance on an alignment-based model means that the combined model is still limited by the constraints of MSAs.

Retrieval-augmented language models have shown impressive results in natural language processing, especially on Question Answering (QA) tasks. These models incorporate a database search as part of the text generation process in order to generate new text conditioned on prototypes found in the database. In this way, they are conceptually similar to the conditional protein language models above. Retrieval-augmented approaches have the advantage of not requiring the entire training corpus to be encoded within the model parameters and the ability to easily integrate new data without retraining by simply adding it to the retrieval database.

### SUMMARY

According to this disclosure, a retrieval-augmented framework leverages a generative protein language model of whole protein families. The approach frames the protein sequence generation problem as a sequence-of-sequences problem to incorporate retrieved-sequence conditioning, thereby providing a fundamentally different paradigm than that employed by current retrieval-augmented models in natural language processing. To this end, a generative protein language model, referred to herein as a Protein Evolutionary Transformer (PoET), is configured and trained on a large set of homologous sequences. By learning to generate sets of related proteins as sequences-of-sequences across very large numbers (e.g., tens of millions) of natural protein sequence clusters, PoET generalizes about evolutionary processes across protein families, and it avoids issues related to conditioning on MSAs. In order to capture conditioning between sequences in an order independent manner (typically, the order of sequences within a family is arbitrary) and to generalize to large context lengths, PoET leverages a transformer layer that models order-dependence between tokens within sequences and order-independence between sequences.

In a representative protein engineering workflow, PoET is configured as a generative model of whole protein families and used for controllable design of protein sequences and variant effect prediction. PoET generates sets of homologous proteins as sequences-of-sequences. In a typical use case, the model is controlled by providing it with a prompt, namely, a set of sequences that represent homologues, family members, or some other grouping of related sequences that represent a protein of interest, and receiving an output from the model. Given a prompt, the model enables various protein engineering workflows, e.g., scoring of arbitrary sequences to predict sequence fitness and rank variants, mapping of fitness of single substitution variants, identifying mutable hotspots and designing combinatorial variant libraries, generating bespoke, high order variants (by sampling from the model), and exploring diverse sequence space of a protein, and the like.

According to a variant embodiment, sometimes referred to herein as PoET-2, a multimodal generative model of protein families is provided to jointly model the sequences and structures of proteins within a protein family. The model is used for controllable protein generation and representation learning. In operation, PoET-2 leverages in-context learning to infer underlying evolutionary constraints that give rise to the family's characteristic sequence features, structural architectures, and/or functional properties. These inferred evolutionary constraints, coupled with a flexible grammar for specifying explicit sequence and structural constraints, enables the controlled generation of novel family members, including variants with optimized or enhanced characteristics relevant to their function. Preferably, PoET-2 is implemented as an encoder-decoder transformer, preferably one encoder, and two decoders. The encoder processes a user-provided prompt comprising a set of proteins and that guides the two decoders toward generating novel proteins with desired characteristics.

In a representative embodiment, the PoET-2 prompt is processed in a fully protein order equivariant manner and includes a context component, and optionally a query component. The context describes a protein family comprising a set of proteins are believed likely to exhibit at least one of the desired characteristics. The query typically is a single, partially specified protein that specifies the sequence and/or structure at only a subset of residues. When used, the query constrains the model to generate only proteins containing those sequence or structural elements. Common use cases of the query include, without limitation, specifying the protein length, the presence of certain peptides, the inclusion of active sites, or the structure of the entire protein backbone (i.e. inverse folding). Together, the context and the query provide the flexible grammar that allows sequence generation to be controlled both implicitly (via the context, which includes example sequences and structures likely to exhibit the desired characteristics), and explicitly (via the query, which specifies the explicit sequence and structure constraints). Prompt engineering via appropriate selection of the context enables the model to focus on the evolutionary, structural and/or functional constraints of only a particular subspace of relevant proteins. The decoders, conditioning on the encoder's output, generate new proteins aligned with the prompted protein family. Preferably, the decoders are complementary to one another, with the first decoder (e.g., an autoregressive decoder) being trained with a causal language modeling (CLM) objective and specialized for generative tasks, and the second decoder (e.g., a bidirectional decoder) trained with a masked language modeling (MLM) objective and specialized in representative learning.

The foregoing has outlined some of the more pertinent features of the subject matter. These features should be construed to be merely illustrative. Many other beneficial results can be attained by applying the disclosed subject matter in a different manner or by modifying the subject matter as will be described.

### BRIEF DESCRIPTION OF DRAWINGS

FIG. 1 depicts a sequence of three protein sequences of different lengths;

FIG. 2 depicts a probability function of a sequence-of-sequences (such as depicted in FIG. 1) that is used by PoET to generate each token in the sequence;

FIG. 3 depicts an inter-sequence relative position encoding scheme used in sequence-of-sequences self-attention within PoET that is invariant to sequence ordering;

FIG. 4 depicts a representative PoET architecture according to this disclosure;

FIG. 5 depicts a first algorithm corresponding to the TieredTransformerEncoderLayer shown in FIG. 4;

FIG. 6 depicts a first model variant, referred to herein as Prefix-LM PoET with encoder-decoder architecture, which is trained on a prefix language modeling objective;

FIG. 7 depicts a second algorithm corresponding to the TieredTransformerDecoderLayer shown in FIG. 6;

FIG. 8 depicts a second model variant, referred to herein as MLM PoET, which is trained on a Masked Language Modeling (MLM) objective, rather than a causal or prefix language modeling objectives;

FIG. 9 depicts a third model variant, referred to herein as Multi-task PoET, which provides for an encoder-decoder prefix-LM PoET additionally trained on an MLM objective;

FIG. 10 depicts a third algorithm, referred to herein as PoETEmbed, by which the PoET model creates useful pre-residue embeddings;

FIG. 11 depicts a first protein engineering workflow, namely, for variant prioritization;

FIG. 12 depicts a second workflow, namely, sampling sequences from the model for generating new sequences that have a same function as a known set of sequences;

FIG. 13 depicts several examples of chaining natural language and protein sequences for prompt engineering for the task of using the model to specify a protein family using natural language descriptions;

FIG. 14 depicts a third workflow, namely, a workflow to facilitate sequence to function learning;

FIG. 15 depicts a fourth workflow, namely, generating a per-residue annotations model using PoET;

FIG. 16 depicts a fifth workflow, namely, generating a structure prediction model using PoET; and

FIGS. 17-20 depict various user interfaces for receiving information to generate prompts for use against the PoET model in various use cases.

FIG. 21 depicts an alternative embodiment of the PoET model, the embodiment referred to herein as PoET-2;

FIG. 22 depicts an input embedding algorithm, referred to herein as Algorithm 4, which embeds a single sequence or a sequence-of-sequences;

FIG. 23 depicts an example of how PoET-2 employs a structure-based attention bias for all attention operations within individual protein sequences in both its encoder and decoders;

FIG. 24 depicts an algorithm, referred to herein as Algorithm 5, for generating protein order equivariant layers;

FIG. 25 is an encoder, referred to herein as Algorithm 6, which operates to encode a prompt x composed of a sequence-of-sequences, and that transforms embeddings by applying n_{layers} of the protein order equivariant layers;

FIG. 26 depicts an algorithm, referred to herein as Algorithm 7, for generating PoET-2's decoder layers;

FIG. 27 is a decoder, referred to herein as Algorithm 8, which operates to decode a single sequence y conditioned on prompt embedding provided by the encoder and using the decoder layers;

FIG. 28 depicts a workflow for an unmask generation mode;

FIG. 29 depicts a workflow for a target homology generation mode;

FIG. 30 is an algorithm, referred to herein as Algorithm 9, for creating per-residue embeddings for the encoder;

FIG. 31 is an algorithm, referred to herein as Algorithm 10, for creating per-residue embeddings for the decoder;

FIG. 32 depicts a workflow for variant prioritization using PoET-2;

FIG. 33 depicts a workflow for creating sequence-to-function predictors using PoET-2;

FIG. 34 depicts a per-residue annotations model using PoET-2;

FIG. 35 depicts structure prediction modeling using PoET-2; and

FIG. 36 depicts the PoET-2 architecture and framework for zero-short and supervised variant effect prediction.

### DETAILED DESCRIPTION

By way of background, the following terms have the following meaning.

A “language model” is a probabilistic model of sequences. In the case of natural language, language models typically describe the probability of sentences or documents. In the case of proteins, they model the probability of amino acid sequences. Being simply probabilistic models, language models can take on many specific incarnations, e.g., from column frequencies in multiple sequence alignments to Hidden Markov Models to Potts models to deep neural networks.

A “generative model” is a model of a data distribution, p(X), joint data distribution, p(X, Y), or conditional data distribution, p(X|Y=y). It is usually framed in contrast to discriminative models that model the probability of the target given an observation, p(Y|X=x). Here, X is observable, for example the protein sequence, and Y is a target that is not observed, for example the protein structure or function. Conditional generative and discriminative models are related by Bayes' theorem. Language models are generative models.

An “autoregressive language model” is a language model that factorizes the probability of a sequence into a product of conditional probabilities in which the probability of each token is conditioned on the preceding tokens

$$p ( {x}_{1} … {x}_{L} ) = {\prod }_{i = 1}^{L} p ( {x}_{i} \vert {x}_{1} … {x}_{i - 1} ) ..$$

Examples of autoregressive language models include k-mer (also known as n-gram) models, Hidden Markov Models, and typical autoregressive recurrent neural network or generative transformer language models. These models are called autoregressive because they model the probability of one token after another in order.

### Protein Evolutionary Transformer (PoET)

In one embodiment, PoET is an autoregressive generative model of the distribution over protein families, where each family is generated as a sequence-of-sequences. Specifically, it models the distribution P(X=x), where x=s₁, s₂, . . . , sₙ is the concatenation of n sequences sᵢ from the same family, and each sequence sᵢ=s_{i,1}, s_{i,2}, . . . , s_{i,Li} is a sequence of Lᵢ amino acids padded by a start and end token. For example, FIG. 1 depicts a sequence of three protein sequences of lengths 4, 6, and 5 with start token denoted by $ and stop token denoted by *.

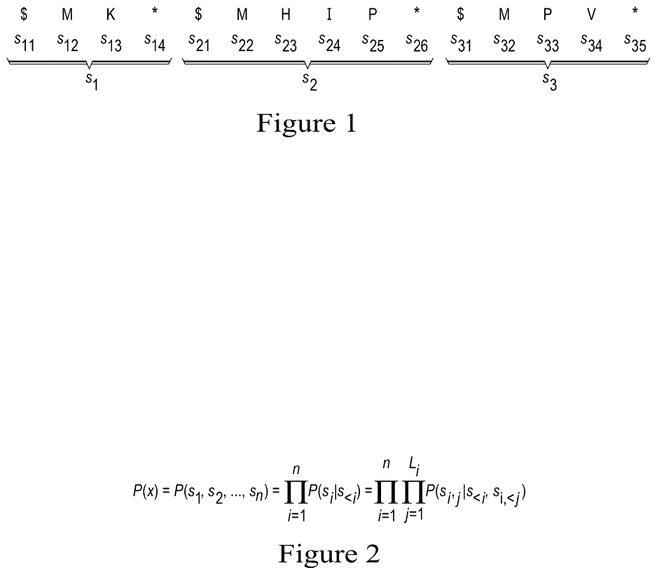

When referring to a sequence-of-sequences, sᵢ, which has one index i, refers to a sequence of tokens, namely the ith sequence in the sequence-of-sequences, whereas s_{i,j}, which has two indices i,j, refers to one token, namely the jth token of the ith sequence. As used herein, x denotes the full sequence-of-sequences. Preferably, PoET generates each token in a sequence x one at a time, decomposing the probability of x as set forth in FIG. 2. The order of the individual sequences in a sequence-of-sequences is arbitrary, and PoET leverages a transformer-based architecture to exploit this order invariance, as is now described.

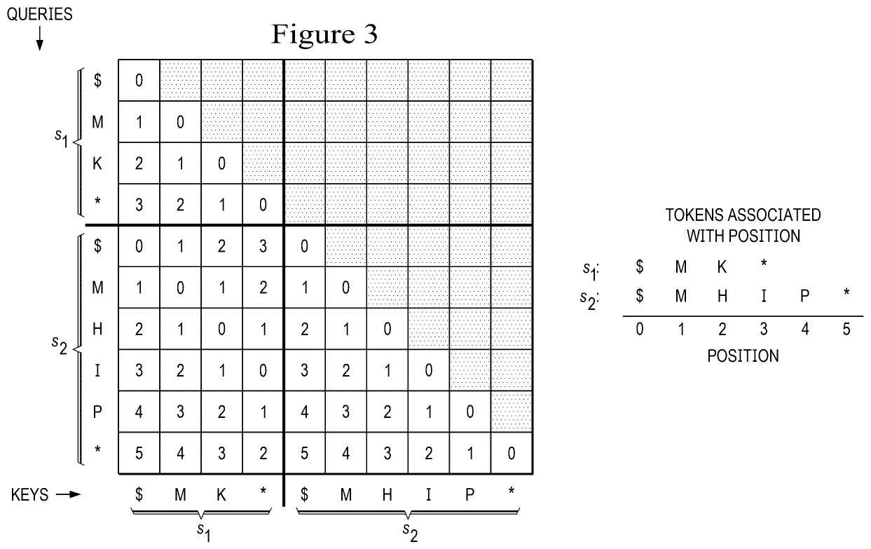

FIG. 3 depicts an inter-sequence relative position encoding scheme used to facilitate sequence-of-sequences self-attention, as will be described. In this example, the first two sequences in the sequence-of-sequences (of FIG. 1) are shown. As depicted on the left side, the relative positions between tokens in the sequence pair are shown, and the right side depicts the resulting tokens associated with a set of absolute positions. The encoding scheme is invariant to sequence ordering, and further details of the scheme are referenced below.

The following provides additional details regarding the Protein Evolutionary Transformer (PoET).

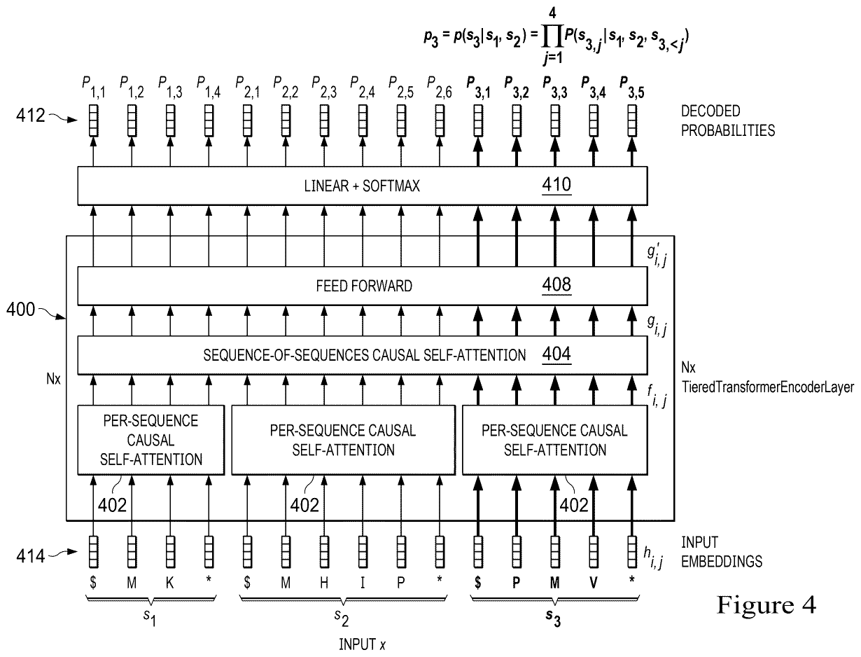

With reference now to FIG. 4, the transformer-based architecture of this disclosure comprises a specialized transformer encoder layer 400 to capture order invariance between sequences while preserving order-dependence between tokens within sequences. In this embodiment, this is accomplished using two attention modules: (i) a within-sequence module 402 in which the representation at each position of each sequence is updated based on attending only to the other tokens within this sequence, and (ii) a between-sequence module 404 in which the representation at each position of each sequence is updated based on attending to all sequences within the sequence-of-sequences. This tiered approach ensures capture of long-range dependencies between sequences and uniquely allows the model to extrapolate to much longer context lengths than used during training, thereby improving sequence generation and performance on downstream tasks. In a particular implementation, the PoET model is a stack of these layers with causal self-attention. As also depicted, outputs from the between-sequence module 404 are applied to a feed forward module 408, which prepares the outputs for further processing, with the result generated by the transformer encoder layer 400 then processed through a Linear+SoftMax module 410 to generate a set of decoded probabilities 412 corresponding to a set of input embeddings 414 received by the model. In particular, a linear layer in module 410 takes the decoded activations and projects them to a size of the vocabulary (as logits). The SoftMax layer in the module 410 takes these logits and generates next-token probabilities. For example, a next predicted token is the argmax of the softmax output.

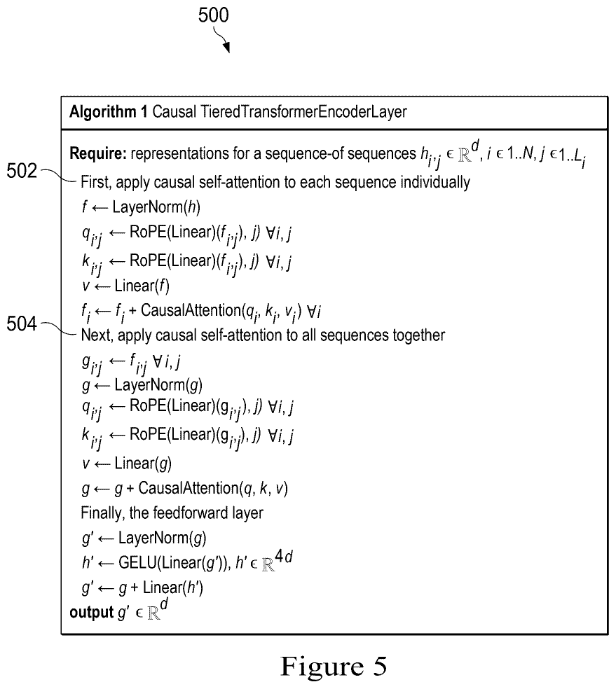

The following provides additional details regarding how the transformer encoder layer operates. Given an input sequence x=s_{i,j}, i∈1 . . . n, j∈1 . . . Lᵢ of amino acids and start/stop tokens, this sequence is first converted into a sequence of continuous embeddings h_{i,j} by mapping each token to its learned embedding:

$${h}_{i , j} = {W}_{si , j} , {s}_{i , j} ∈ AA ⋃ \{ START , STOP \} , {W}^{∈} {R}^{\vert {AA}_{⋃} \{ START , STOP \} \vert \times d}$$

where AA is the set of 20 standard amino acids, and W is a matrix of learnable embeddings of dimension d. Next, the embeddings h_{i,j} are transformed by N layers of a first algorithm (Algorithm 1), which is referred to herein as a Causal TieredTransformerEncoderLayer, and which is specified by the listing 500 in FIG. 5. As noted above, this transformer is a specialized layer for processing a sequence-of-sequences that is invariant to the order of the individual sequences, and that extrapolates to context lengths substantially longer than the training context length.

The TieredTransformerEncoderLayer 500 is composed of two phases. In the first phase, at 502, causal self-attention is applied independently to each sequence hᵢ of the input sequence-of-sequences, transforming them into new sequences ƒᵢ=PerSequenceSelfAttn(hᵢ). This is the operation of the within-sequence module 402 in FIG. 4. Preferably, relative positional information is encoded by applying Rotary Positional Encodings (RoPE) to the queries and keys before applying self-attention; the absolute position for ƒ_{i,j} is j.

The second phase, at 504, applies causal self-attention to the entire sequence-of-sequences by concatenating the individual ƒᵢ from the previous layer into one sequence before applying self-attention: g_{i,j}=SequenceOfSequenceselfAttn([ƒ_{<i};ƒ_{i,<j}]). In order to make self-attention in this phase invariant to sequence order, the inter-sequence relative positional encoding scheme of FIG. 3 is used, namely: for g_{i,j} the absolute position is j. Just as in the first phase, the absolute position for tokens in the ith sequence gᵢ is independent of the position iof the sequence in the sequence-of sequences. Thus, the positional information encoded by RoPE in this layer alone does not distinguish between the positions of tokens in different sequences. For example, the relative position between the first token of the first sequence ƒ_{1,1} and the first token of the second sequence ƒ_{2,1} is 0. The fact that these two tokens come from two different sequences is encoded by the first phase, which operates on the two sequences independently. This inter-sequence relative positional encoding scheme has several useful properties: it encodes the fact that amino acids at similar absolute positions in homologous proteins are more likely to be drawn from the same distribution, and it limits the maximum relative position encoding needed to the number of tokens in an individual protein sequence, rather than the number of tokens in a sequence-of-sequences, allowing the model to generalize to longer sequences-of-sequences than seen during training.

Lastly, and referring back to FIG. 5, at 504, the output from the last TieredTransformerEncoderLayer, g_{i,j}, is decoded into token probabilities by applying a linear transformation P(s_{i,j}|s_{<i},s_{i,<j})=p_{i,j}(s_{i,j})=Linear(g_{i,j}). Here, p_{i,j} is a vector of probabilities, one for each distinct token∈AAU{START,STOP}, and p_{i,j}(s_{i,j}) is the probability of the token s_{i,j} according to p_{i,j}.

### Training

The PoET model is trained on large sets of homologous sequences. In one example training, PoET is trained on 29 million sets of related sequences, with each set corresponding to a sequence in UniRef50 Version 2103, and containing all its homologs in UniRef50 found using the Diamond search tool. Any sets with fewer than 10 homologs are removed. To avoid overfitting on promiscuous sequences which may belong to a large number of sets, each set is sampled with weight inversely proportional to the size of the set (“inverse count” sequence weighting).

To find homologs in UniRef50 using Diamond, an all-against-all search was carried out, and that search returns, for each sequence in UniRef50, a set containing all its putative homologs in UniRef50. Each such set is a “Diamond-UniRef50 Cluster.” Diamond was used over other homology search tools due to its high performance (>100× speed of BLAST).

To form a training example, sequences are then sampled without replacement from a “Diamond-UniRef50 Cluster” until the total number of tokens reached a predetermined limit, and then concatenated to form a sequence-of-sequences. Each sampled UniRef50 sequence is replaced with a UniRef100 sequence by sampling a random UniRef100 sequence from the same UniRef50 cluster as the UniRef50 sequence being replaced. The UniRef100 sequences are randomly sampled with weight inversely proportional to the size of the UniRef90 clusters to which they belong. Preferably, each UniRef100 sequence is sampled at most once in each sequence-of-sequences.

As a final data augmentation, preferably the order of the tokens in a sequence-of-sequences is reversed with probability 50% (i.e. all sequences in a sequence-of-sequences are ordered from either N-terminus to C-terminus only or C-terminus to N-terminus only). This augmentation has been shown to improve the performance of other protein language models.

Following this sampling procedure, the order of sequences in a sequence-of-sequences is random, which promotes order invariance.

### Loss Function

In the above-described example, the model is trained to minimize the negative log likelihood of the next token:

$$- log P ( x ) = - {{\sum }_{i = 1}}^{n} {\sum }_{j = 1}^{{L}_{i}} log P ( {s}_{i , j} \vert {s}_{i , < j} )$$

Preferably, the model is also trained to predict the next token with the order of all tokens (within a single protein sequence) reversed and during inference, compute model likelihoods of sequences in their original order and in reverse order.

### Variant Models

The loss function described above is known as a causal language modeling loss function because the probability of the next token is predicted based on only past tokens, without looking at any future tokens. This loss function is particularly useful for sequence generation tasks because it decomposes the probability of a sequence such that it can be generated one token at a time from beginning to end. When, however, the model is not being asked to generate a sequence this causal restriction is needlessly restricting. For example, when prompting the model by providing it a sequence of sequences related to the sequence that is desired to be generated, the representation of the first sequence is independent of the second sequence, even though information about the second sequence may be helpful for building a better representation of the first sequence. A prefix language modeling (Prefix-LM) objective solves this issue by allowing the model to attend fully to a prefix of a sequence, in this case, the part of the sequence that corresponds to the prompt.

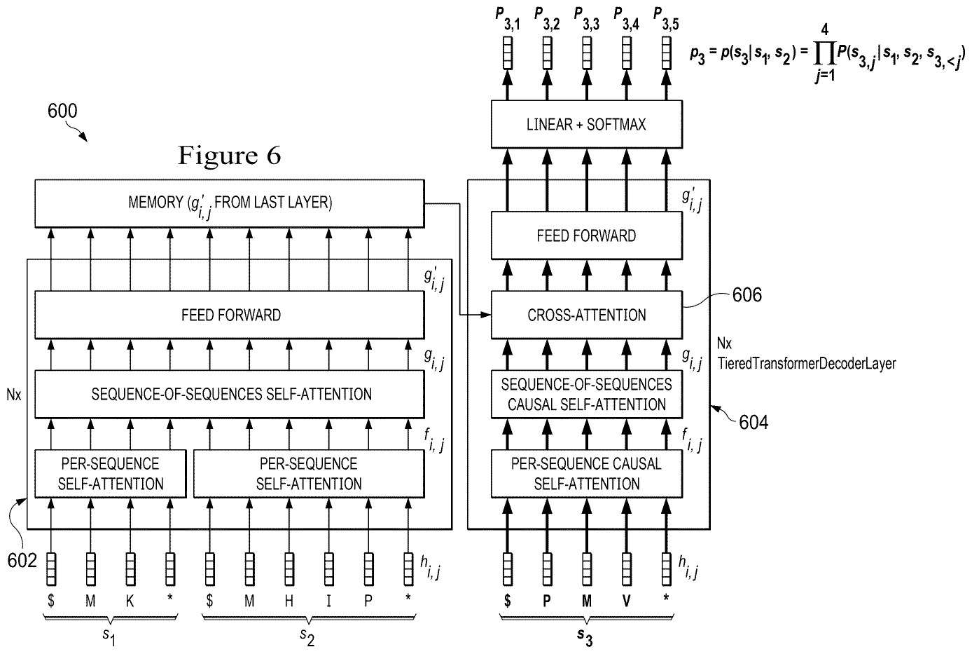

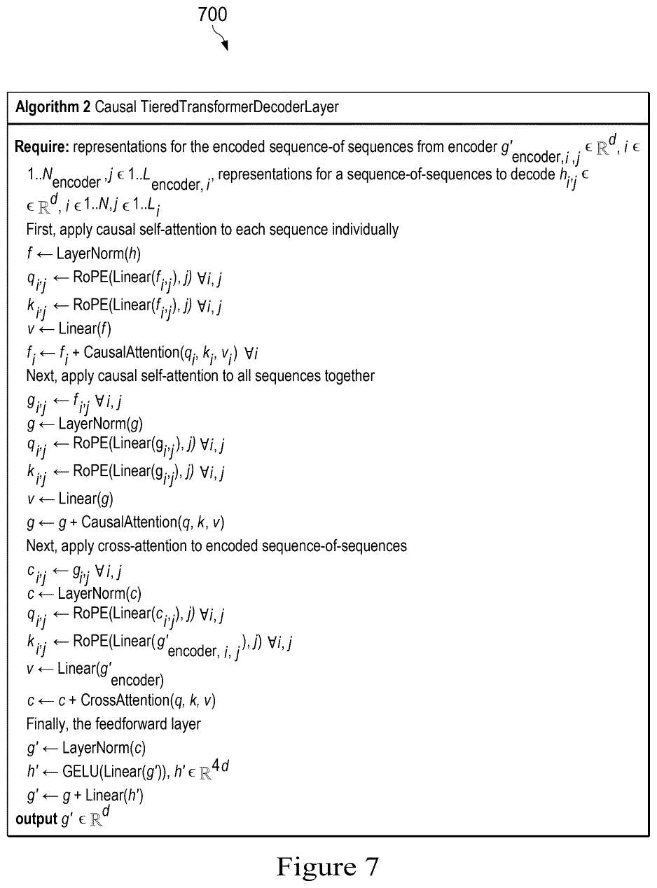

Thus, and with reference now to FIG. 6, a first variant model, which is referred to herein as Prefix-LM PoET, is depicted as reference numeral 600. In this example (with the sequence-of-sequences), the first two sequences are processed in an encoder portion 602 of the transformer, and the next sequence is process in a decoder portion 604. As can be seen, the decoder portion 606 also includes an additional cross-attention function 606 that receives the output of the encoder. The cross-attention function 606 applies cross-attention to encoded sequence-of-sequences. More specifically, the Prefix-LM PoET variant model implements a second algorithm (Algorithm 2) that is depicted in the listing shown in FIG. 7. This algorithm is referred to as TieredTransformerDecoderLayer 700 due to the inclusion of the decoder portion 604, and to differentiate from the TieredTransformerEncoderLayer 500 that was depicted in FIG. 5 (for the regular PoET model).

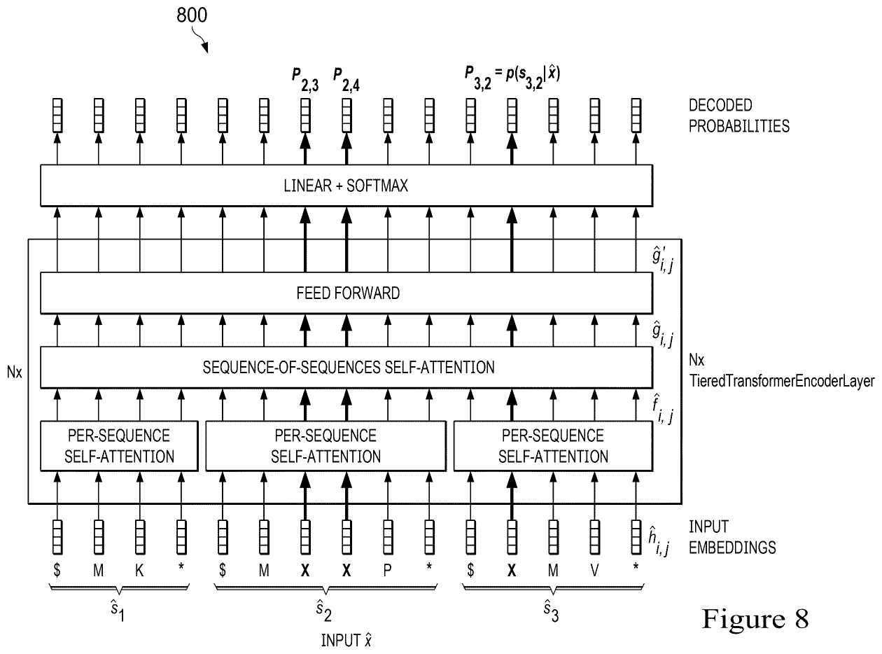

Referring now to FIG. 8, a second variant model, which is referred to herein as MLM PoET, is depicted as reference numeral 800. MLM PoET is a variation of the PoET model (of FIG. 4) and that is trained on the masked language modeling (MLM) objective, rather than the causal or prefix language modeling objectives for causal and prefix-LM PoET. The MLM objective replaces a subset of the tokens in the input sequence with either a special mask token or a random token from the vocabulary, and then asks the model to predict the replaced token. In the context of PoET, the corresponding loss function is:

$$- log P ( x , \hat{x} , m ) = - {{\sum }_{i = 1}}^{n} {\sum }_{j = 1}^{{L}_{i}} {{m}_{i , j}}_{} log P ( {s}_{i , j} \vert \hat{x} )$$

In this function, x\hat{ }=s\hat{ }₁,s\hat{ }₂, . . . , s\hat{ }ₙ is the masked version of x, and m_{i,j} are random variables indicating whether or not the jth token of the ith sequence is masked. In one example embodiment, 15% of tokens are masked, and among masked tokens, 90% of tokens are replaced with mask tokens, and 10% are replaced by random tokens. These proportions can be chosen differently and even varied during training.

The MLM objective as implemented in FIG. 8 is useful because it allows the model to attend to all tokens in each sequence, rather than only causal attention (for the full sequence in causal language modeling and for the suffix in prefix language modeling).

The Causal PoET architecture in FIG. 4 can adapted to perform MLM by simply replacing “Causal Self-Attention” in (modules 402 and 404) with “Self-Attention.”

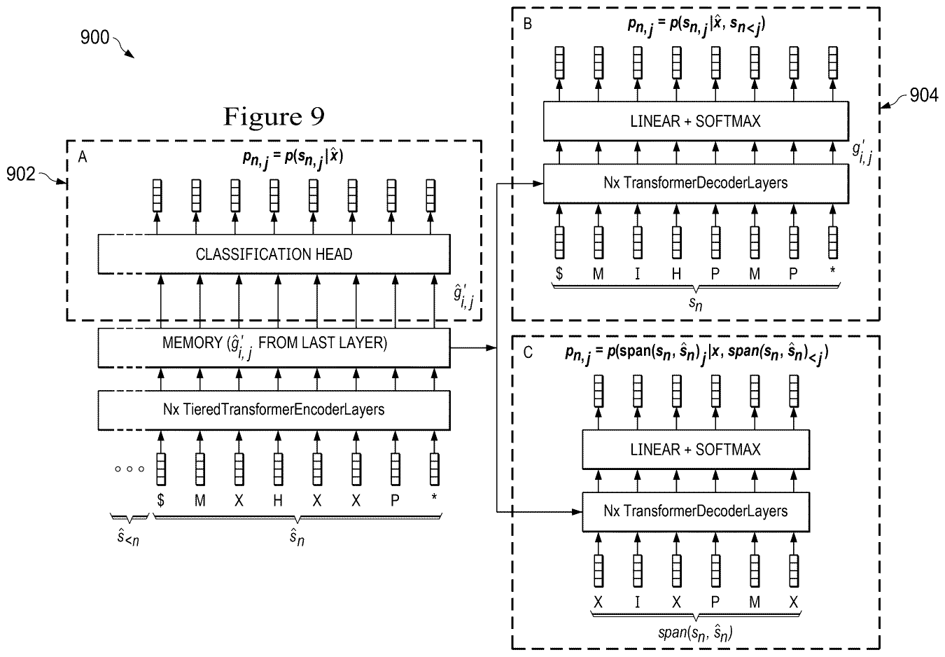

The encoder-decoder variant of Prefix-LM PoET depicted in FIG. 6 (and described by Algorithm 2 in FIG. 7) can be enhanced to perform masked language modeling in addition to prefix language modeling by adding an additional decoder that is conditioned on the memory outputted by the encoder portion and training it to optimize the masked language modeling objective. This variant model is depicted in FIG. 9 as reference numeral 900. This model is referred to herein as Multi-task PoET: Encoder-Decoder Prefix-LM PoET with MLM-like objectives. As depicted in FIG. 9, there are several ways to accomplish this, e.g., option A, which involves adding a classification head 902 to predict the original identities of masked tokens and train the model to optimize the MLM PoET model loss function described above. Option B involves adding a regular Transformer decoder 904 that autoregressively decodes each individual sequence in the sequence-of-sequences by performing cross-attention with the subsequence of the encoder memory that corresponds to the sequence being decoded. For option B, the loss function is similar to the MLM PoET model loss function, except that the model is allowed to condition on the true values of all previous tokens regardless of whether or not they are masked in the encoder. Thus, for Option B, the loss function is:

$$- log P ( x , \hat{x} , m ) = - {{\sum }_{i = 1}}^{n} {\sum }_{j = 1}^{{L}_{i}} log P ( {s}_{i , j} \vert \hat{x} , {s}_{i , < j} )$$

Referring back to FIG. 9, a third option, Option C, is the same as Option B except that in the decoded sequence, all consecutive unmasked tokens are replaced with a single mask token. For example, suppose that the original sequence is s=$MIHPMP*, and the masked sequence is s\hat{ }=$MXHXXP* (masked tokens are denoted by X). Then, and in Option C, the sequence to decode is given by a “span function” span(s,s\hat{ })=XIXPMX. The corresponding loss function is:

$$- log P ( x , \hat{x} , m ) = - {{\sum }_{i = 1}}^{n} {\sum }_{j = 1}^{L ( span ( {s}_{i} , {\hat{s}}_{i} ) )} log P ( {span ( {s}_{i} , {\hat{s}}_{i} )}_{j} \vert \hat{x} , {span ( {s}_{i} , {\hat{s}}_{i} )}_{< j} )$$

In the above loss function, the function L(⋅) gives the length of the input sequence.

The following describes obtaining homology augmented per-residue embeddings from PoET models. An embedding of a residue in a protein sequence is a real valued vector representing the unique context of that residue in its protein sequence. The corresponding embedding function is the function that transforms the residue into the embedding. Such an embedding function is useful when it places residues of similar contexts close to each other, and residues of differing contexts far away from each other (e.g. as measured by Euclidean distance). For example, two residues in two different protein sequences that both participate in the catalysis of the same chemical reaction may be considered similar, while a residue that does not participate in any catalysis may be considered dissimilar. The specific definition of the “context” of a residue is application dependent; the ideal embedding function works for any such definition of the context, or is able to adjust the embedding based on the definition of the context.

PoET models naturally create useful per-residue embeddings. In particular, these models include an embedding function that maps residues to a corresponding outputs

$${g}_{i , j}^{′}$$

of the “TieredTransformerEncoderLayer” (see, e.g., Algorithm 1) within the PoET models. These outputs are labeled as either

$${g}_{i , j}^{′} or {\hat{g}}_{i , j}^{′}$$

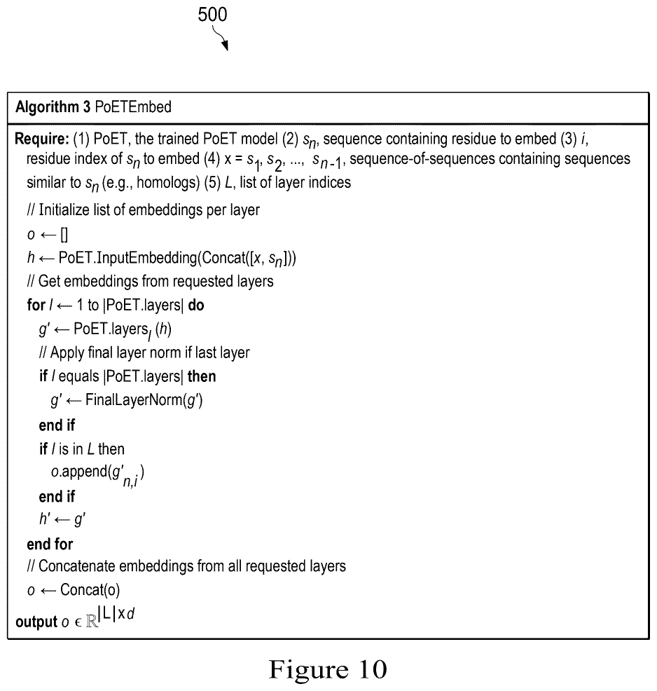

in the PoET architecture diagrams (FIGS. 4, 6 and 8-9). FIG. 10 depicts a third algorithm (Algorithm 3), referred to herein as PoETEmbed, for encoder-only PoET models. Embeddings can also be obtained from the decoder of (1) the encoder-decoder variant of Prefix-LM PoET and (2) variants A and B of Multi-task PoET by using the output of the “TieredTransformerDecoderLayer” or TransformerDecoderLayer” instead (Algorithm 2, FIG. 6). The decoder of Option C of the Multi-task PoET is not useful for this purpose.

The output of PoETEmbed (Algorithm 3) can be adjusted for different contexts by changing the sequence-of-sequences x, also called the “prompt”, that PoET is conditioned on. Examples of different prompting methods are set forth below. Because the prompt generally contains protein sequences homologous to the protein family of interest, the embeddings obtained from PoET models are conveniently described as “homology augmented.” This homology augmentation differentiates PoET from other existing protein language models, which do not directly condition on homologous sequences and whose embeddings cannot be adjusted for different contexts.

### Applications and Use Cases

The PoET models described herein facilitate various protein engineering workflows. Several example workflows are now described.

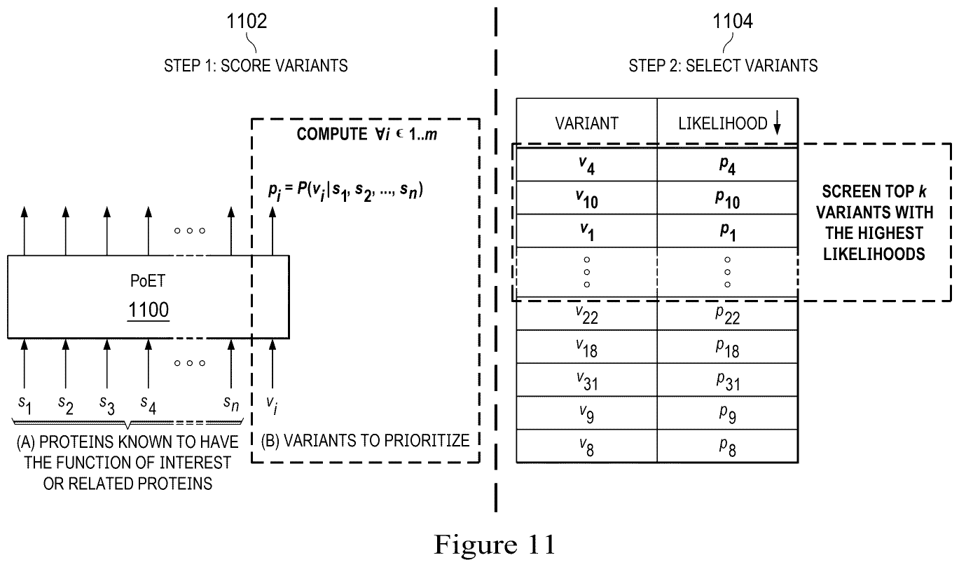

A first application/use case is variant prioritization, which is depicted in FIG. 11. Variant prioritization is applicable to all PoET variants, except MLM PoET. For Multi-task PoET variants, the decoder trained with the Prefix-LM objective is used. Protein variant fitness prediction is the task of assigning a score to each sequence in a set of variants {v₁,v₂, . . . , vₙ} of a target sequence t that accurately reflects the relative fitness of the variants. A protein variant vᵢ can be any sequence with a limited number of substitutions, insertions, and/or deletions relative to the target t that the experimenter believes may have improved fitness. Fitness refers to the value of any property of a protein sequence related to function that the experimenter is interested in optimizing e.g. thermostability, enzymatic activity, etc. As depicted in FIG. 11, the variant prioritization workflow with respect to the model 1100 involves a first step 1102 to score variants, and a second step 1104 to select the variants based on the scoring. In one embodiment of this workflow, fitness prediction with PoET works as follows.

In this embodiment, PoET predicts the fitness of a variant as the conditioned log-likelihood of the variant vᵢ given a set of sequences S homologous to the target t:

$${\hat{F}}_{i} ( S = \{ {s}_{1} , … , {s}_{m} \} ) = log P ( {v}_{i} \vert {s}_{1} , {s}_{2} , … , {s}_{m} ) = {\sum }_{j = 1}^{{L}_{i}} log P ( {v}_{i , j} \vert {s}_{1} , {s}_{2} , … , {s}_{m} , {v}_{i , < j} )$$

In this example, the set S is retrieved by searching a large database of proteins such as UniRef100 for sequences homologous to t. The ColabFold protocol is used for retrieval, but this is not a limitation. The homologs form a diverse set of sequences that define the protein family of interest. By conditioning on these sequences, PoET infers evolutionary constraints on the protein family to improve fitness prediction. The sequences are conditioned on in an arbitrary order. In this example, the homologous sequences were subsampled and filtered to a reasonable context length for efficient inference, and conditional log-likelihoods are computed from different ensembles of homologous sequences. The final fitness prediction scores are obtained by averaging the conditional log-likelihoods across subsamples of the full set of retrieved homologous sequences:

$${\hat{F}}_{ensemble , i} ( S ) = \frac{1}{{N}_{ensemble}} {\sum }_{j = 1}^{{N}_{ensemble}} {\hat{F}}_{i} ( {S}_{j} ⊂ S )$$

Using the above-described technique, PoET has been shown to provide state-of-the art performance on variant fitness prediction. This performance demonstrates that PoET assigns higher likelihood to regions of sequence space with high fitness variants.

Accordingly, and as another example protein engineering workflow, PoET is used to directly generate high fitness variants belonging to a protein family and, in particular, by conditioning PoET on sequences from the protein family and sampling from the resulting conditional distribution. Amino acids are sampled using various techniques, such as top k sampling, nucleus sampling, or beam search. Direct generation of variants makes the exploration of higher order mutants computationally tractable as sequence space grows exponentially large with number of mutations, making it impossible to explore this space by scoring all such variants. This application/use case (direct generation of novel protein sequences with user specified functions) is applicable to all PoET variants except MLM PoET. For Multi-task PoET variants, the decoder trained with the Prefix-LM objective is used.

Further details of using PoET to facilitate function-specific variant fitness prediction and sequence generation via prompt engineering are now described. As noted above, a PoET model as has been described has the ability to predict the “general” fitness of a protein variant, where “general” fitness can refer to any property that is related to the function of the protein. In practice, it is also desired to optimize specific properties of a protein. Because many properties of interest are correlated and together contribute to the general fitness of a protein (e.g. a more thermostable variant is also more likely to have higher expression or enzyme activity), optimizing general fitness is likely to optimize the true properties of interest, albeit indirectly. As noted above, PoET is useful for protein engineering because it is able to successfully predict general fitness by conditioning on and inferring evolutionary constraints from a diverse set of homologs of the target protein. By extension, and according to a further aspect, PoET is used to optimize specific properties of interest, preferably by conditioning on only the subset of relevant homologs that are known to or are predicted to display the specific properties of interest.

To this end, and based on experimental results, PoET is able to learn function-specific evolutionary constraints for the target protein and property of interest, e.g., in the chorismate mutase indels dataset from ProteinGym. The dataset contains measurements of the catalytic activity, in E. coli, of 1130 natural chorismate mutase sequences, and 1618 designed chorismate mutase variants. The natural sequences are comprised of the target protein, a chorismate mutase found in E. coli, and homologs of the target protein found using the PSI-BLAST program for sequence search. The designed variants were selected by Monte Carlo sampling from a Potts model trained on an MSA of the natural sequences. This data presents an ideal scenario for selecting the subset of most relevant homologs; the subset of natural homologs that are measured to be functional are the ones selected. In the absence of such data, one could instead use predictions from another model, or other relevant known attributes of the sequences e.g. to optimize for activity at high temperatures, select only the homologs from thermophiles.

On the chorismate mutase dataset, it was found that the catalytic activity of designed chorismate mutase variants is better predicted when PoET is conditioned on only the subset of functional natural sequences rather than all natural sequences (Δρ=0.2). In fact, PoET conditioned on functional natural sequences outperforms fully supervised methods, including a Gaussian process trained on mean embeddings from a BERT-like protein masked language model (Δρ=0.06). Such embeddings have been shown to be highly predictive of a variety of protein properties and provide a strong baseline. These fully supervised methods are trained on more data than PoET because they train on the measured catalytic activities of all the natural sequences, whereas PoET is simply conditioned on positively labeled natural sequences and does not have access to the measured activities. This enables PoET to be used with assays that only measure binary endpoints rather than continuous values.

Based on these results, PoET thus is shown to be useful for function-specific sequence generation. In the above scenario, and conditioned on the functional natural sequences, PoET was then to generate 1000 novel putative chorismate mutases using nucleus sampling with p=0.9.

Generalizing, and with reference to FIG. 12, a PoET model is used to generate new sequences that are related to (i.e., has the same function as) a known set of sequences. This is accomplished by prompting the PoET model with the known set of sequences, and then sampling from the model by using the predicted next token probabilities to determine the sequence of amino acids one at a time. Amino acids are sampled using various techniques, such as top k sampling, nucleus sampling, or beam search. FIG. 12 depicts the process for the PoET model 1200. In this example, it is desired to have the model generate the sequences related to the proteins with sequences MI and MHIP. The prompt 1202 ends on a start token to indicate that the model should predict the probability for the first amino acid of a new sequence. As depicted, and based on the prompt 1202, the model has predicted the probability distribution 1204 of the first amino acids. Only a subset of amino acids in the distribution are shown for brevity. At step (2), an amino acid then chosen as the first amino acid by sampling from this distribution. Processing then continues at step (3) by adding the chosen amino acid to the prompt and re-applying the prompt to the model. At step (4), the next token is sampled. At step (5), the above-described process is repeated until at step (6) a stop token is reached. This completes the sequence generation process for the prompt.

In the above example, the kind of proteins that PoET is prompted to generate is based on showing PoET examples of proteins that exhibit the function of interest, without directly specifying exactly what the function is. According to a further aspect of this disclosure, PoET can be extended to allow the function to be specified by natural language. In this embodiment, this is accomplished by extending the vocabulary to include tokens from natural language, and then prefixing protein sequences with a natural language description of the protein. The following example illustrates this approach.

In this example, the prompt begins with a natural language description of a protein, denoted by a special start token [SN], followed by the sequence of the protein described, denoted by the regular start token [S]. Next, the PoET model is provided with the natural language description of the protein that it is desired for the model to generate, and which is similar to but not exactly the same as the first. Finally, the prompt ends with the regular start token to indicate that the model should begin generating a new sequence:
Prompt: [SN] A PETase from a thermophillic organism. [E][S] LVDKYYET [E] [SN] A PETase from a mesophillic organism. [E][S]

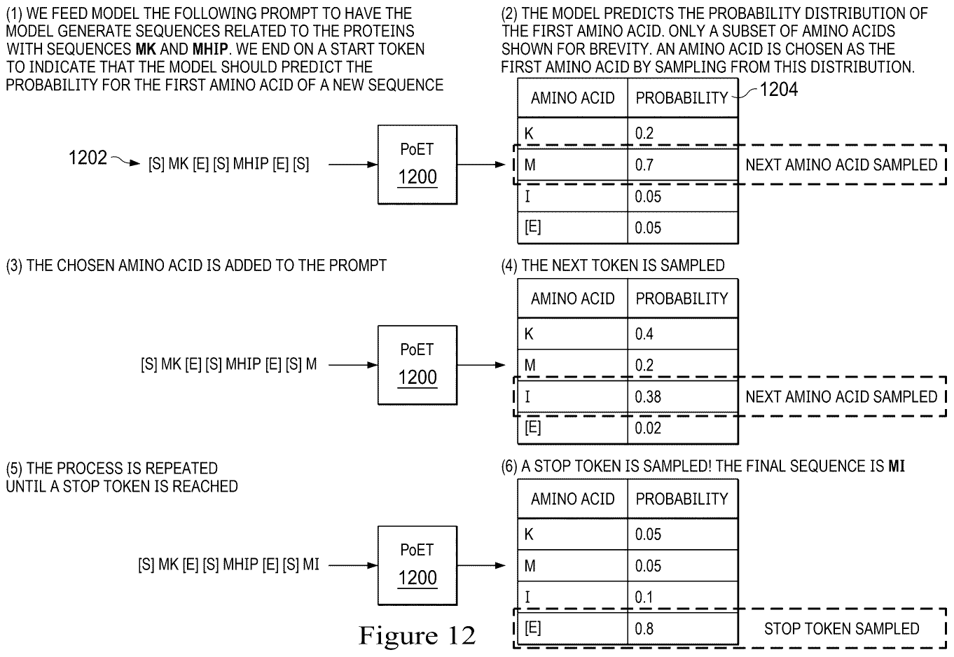

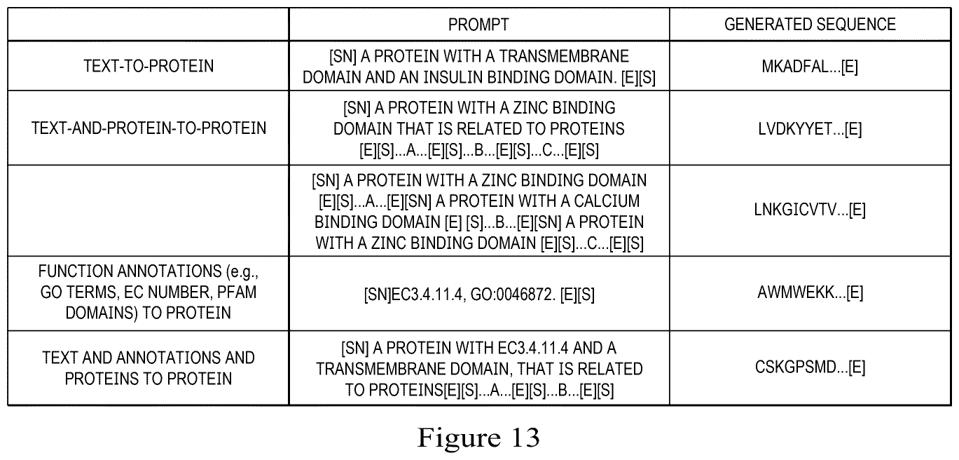

The generated sequence from this prompt is AWMWEKK[E]. Additional examples of this natural language-supported prompting are shown in FIG. 13. By chaining natural language and protein sequences in this way, the model is informed of what properties that the input sequences are known to have, and what properties the sequences that are desired to be generated have, and which may overlap only partially with the properties of the other properties. In the context of the PoET model, each sequence composed of a natural language description and a protein sequence should be considered one sequence in a sequence-of-sequences, and such natural language descriptions can be prefixed to sequences corresponding to both free sequence generation and sequence infilling tasks, which are now described.

The following provides additional details regarding direct generation of novel protein sequences with user specified functions. In one embodiment, the protein family is specified using functional measurements. In particular, functional measurements of properties of interest can be included with each protein sequence (or some of them) in a prompt. This allows the model to learn the relationship between protein sequences and properties of interest, and allows the user to generate sequences with specific property values. Functional measurements can be omitted if they do not exist. For example, the following example prompt specifies the measured “Activity” before each sequence, and requests that the model generate a sequence with “Activity” equal to 5:

$$[ SN ] Ac tivity : - 1 [ E ] [ S ] DYET [ E ] [ SN ] Ac \text{} tivity : N / A [ E ] [ S ] LYYEA [ E ] [ SN ] Ac tivity : 5 [ E ] [ S ]$$

In another embodiment, the protein family is specified using structure. To condition on structure, the structure of each protein sequence can be included in the prompt, e.g., as 3D structure coordinates, or embeddings from a 3D structure embedding model such as an inverse folding model. An example of this type of prompt is as follows:
[SN]<structure coordinates or embeddings of following sequence>[E][S]DYET[E]

$$[ SN ] N / A [ E ] [ S ] LYYEA [ E ]$$

[SN]<structure coordinates or embeddings of requested sequence>[E][S]

In another variant embodiment, the protein family is specified with multiple modalities (e.g., natural language, functional measurements, structure, etc.) by simply providing the data from each modality of interest before the protein sequence in the prompt.

Sequence infilling is the task of designing the amino acid sequence for only a part of a protein, while keeping other parts of the protein constant. This is a straightforward way to preserve protein function in the constant regions with high likelihood. Example applications include antibody CDR design and domain linker design.

To adapt PoET to perform sequence infilling, a prompt is modified as follows. A new start token [SI] is introduced to denote the infilling task. A sequence with the regions to infill masked out by a masking token are then specified. With this modified prompt, the model is then used to generate the complete sequence. In the example below, the model is prompted to infill the two regions of the sequence MK_TA_T denoted by the masking token _. After the normal start token [S], the model generates the infilled sequence, which replaces the masking tokens with sequences of amino acids. The infilled amino acids set off by asterisks but are not different from the normal amino acids:
Prompt: [SI] MK_TA_T [E] [S]

The generated sequence for this prompt is MK*SRA*TA*HK*T. In the PoET context, each such sequence composed of a masked and an unmasked sequence should be considered one sequence in a sequence of sequences. PoET can be trained to perform both sequence infilling and free sequence generation simultaneously by simply training on sequences of sequences that contain both the free generation sequences, denoted by the regular start token [S], and the sequence infilling sequences, denoted by the start token [SI].

Still another application/use case for the PoET architecture is Supervised Learning with homology augmented embeddings. In particular, the following subsections describe how homology augmented embeddings obtained from PoET via Algorithm 3 in FIG. 10 are used to develop improved supervised learning models that address other challenges in protein engineering.

Sequence to Function Learning. The aim of sequence to function learning is to create a mathematical model that predicts the ability of a protein to carry out its functions by learning from an existing dataset mapping protein sequences to quantitative measurements of those functions. Such predictive models can be used in black-box optimization algorithms to propose new hypotheses for protein sequences with enhanced function that can be validated in the lab. Per-residue PoET embeddings can be used to create high quality sequence to function models by fitting machine learning models to predict measurements of function from the per-residue PoET embeddings of protein sequences. According to this aspect, each protein sequence is mapped to a sequence of per-residue PoET embeddings, one for each residue of the protein sequence. Then, each sequence of per-residue embeddings is reduced to a fixed length vector. Examples of such reduction functions include mean pooling, taking the embedding of the last residue of the protein sequence only, and computing a singular value decomposition (SVD). Finally, any supervised machine learning algorithm can be used to learn a function ƒ mapping the reduced embeddings to measurements of functions y\hat{ }ᵢ

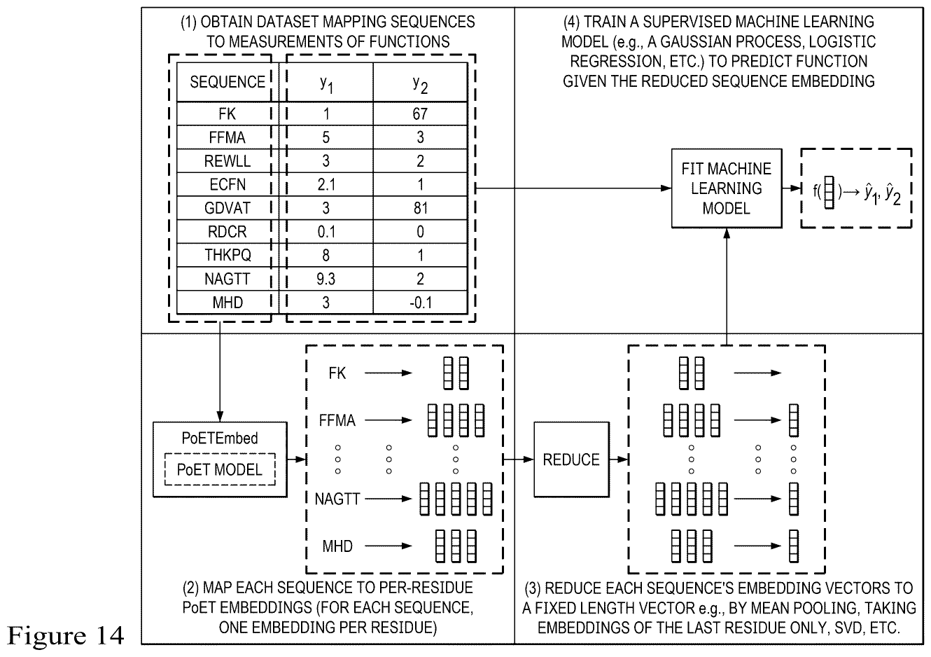

This process is illustrated in FIG. 14. As depicted, in step (1) a dataset mapping sequences to measurements of functions is obtained. At step (2), each sequence is then mapped to per-residue PoET embeddings. At step (3), each sequence's embedding vectors are reduced to a fixed length vector, e.g., by mean pooling, taking embeddings of the last residue only, SVD, or the like. At step (4), and given the reduced sequence embedding, a supervised machine learning model (e.g., a Gaussian process, logistic regression, or the like) is trained to predict the function. If the reduction in Step 2 and the machine learning model learned in Step 3 are differentiable with respect to their inputs, then the entire process (Steps 1-3) as depicted can be learned end-to-end, meaning that the parameters of the PoET model used to create the per-residue embeddings can be finetuned simultaneously via backpropagation.

Per-Residue Sequence Annotation. Per-residue sequence annotation is the task of annotating a set of properties for each residue in a protein sequence. Examples of such properties include secondary structure, transmembrane, torsion angles, disorder, and binding sites. PoET models can be applied to per-residue sequence annotation by adding classifier and/or regression head(s) on top of per-residue PoET embeddings, and then finetuning the resulting model on a dataset containing annotations of the properties of interest. A “classification and/or regression head” refers to a neural network that takes as input the outputs of another neural network (the outputs could be the final outputs of the neural network, and/or internal ‘hidden’ states), such as a pretrained language model, and output predictions to classification and/or regression tasks. Classification and/or regression heads can have multiple outputs, and each output is a prediction for either a classification or regression task, and the outputs together can cover both classification and regression tasks simultaneously. Any existing finetuning technique can be used. “Finetuning” as applied to a neural network means further training (i.e., adjusting the parameters) of a neural network that has already been train on some task, and finetuning may be carried out on new task(s) with different loss function(s).

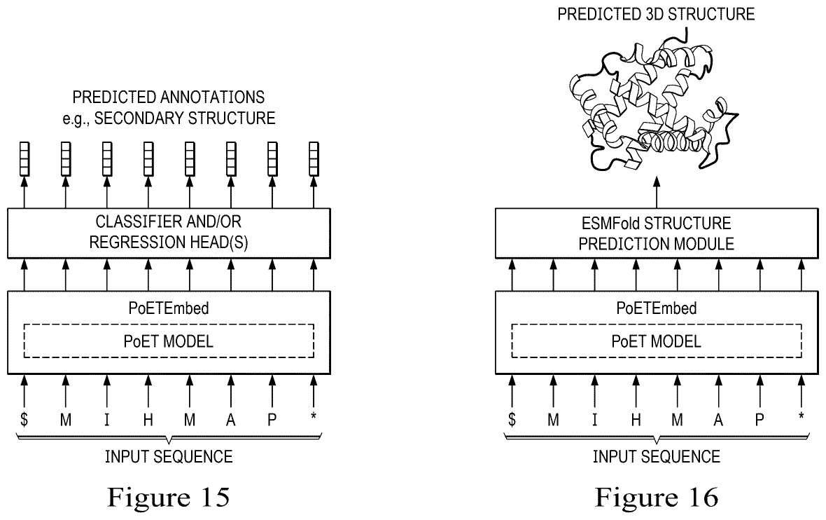

FIG. 15 illustrates a per-residue sequence annotation model utilizing a PoET model.

Another use case for PoET is the task of predicting the 3D structure of a protein sequence. In known techniques, ESMFold currently provides a deep learning-based method to predict protein structure from its amino acid sequence. ESMFold uses a large protein language model for this purpose In this embodiment, PoET models are used for 3D structure prediction by replacing ESM2 per-residue embeddings (used in existing ESMFold approaches) in the ESMFold model with PoET per-residue embeddings, and training the resulting model using the same strategy as used in ESMFold. FIG. 16 illustrates such a model.

Summarizing, PoET is a Transformer-based autoregressive generative model of whole protein families. By framing family generation as a sequence-of-sequences generation problem, we are able to train across tens of millions of protein sequence clusters to encode fundamental rules of protein evolution into the PoET model. This enables the model to generalize to protein families unseen during training and extrapolate from small numbers of conditioned-upon sequences. The sequence-of-sequences generative framework allows PoET to be used as a retrieval-augmented language model, generating new sequences conditioned on a set of sequences representing the family or other properties of interest. PoET has also been demonstrated to improve over other protein language models and evolutionary sequence models for variant fitness prediction across a wide range of deep mutational scanning datasets. PoET also enables efficient sequence generation and the generative distribution can be controlled via conditioning. Phage lysozyme- and chorismate mutase-like sequences sampled from PoET are novel and predicted to fold with high confidence. PoET can be backed by other sequence databases and naturally improves as databases grow without the need for retraining.

The techniques herein have significant advantages. As has been described, PoET is implemented as a retrieval-augmented protein language model by conditioning the model on sequences from any family of interest. This allows PoET to be used with any sequence database and to incorporate new sequence information without retraining. PoET is a fully autoregressive generative model, able to generate and score novel indels in addition to substitutions, and it does not depend on MSAs of an input family, removing problems caused by long insertions, gappy regions, and alignment errors. By learning across protein families, PoET extrapolates from short context lengths, thereby allowing it to generalize well even for small protein families. In addition, PoET can be sampled from and used to calculate the likelihood of any sequence efficiently.

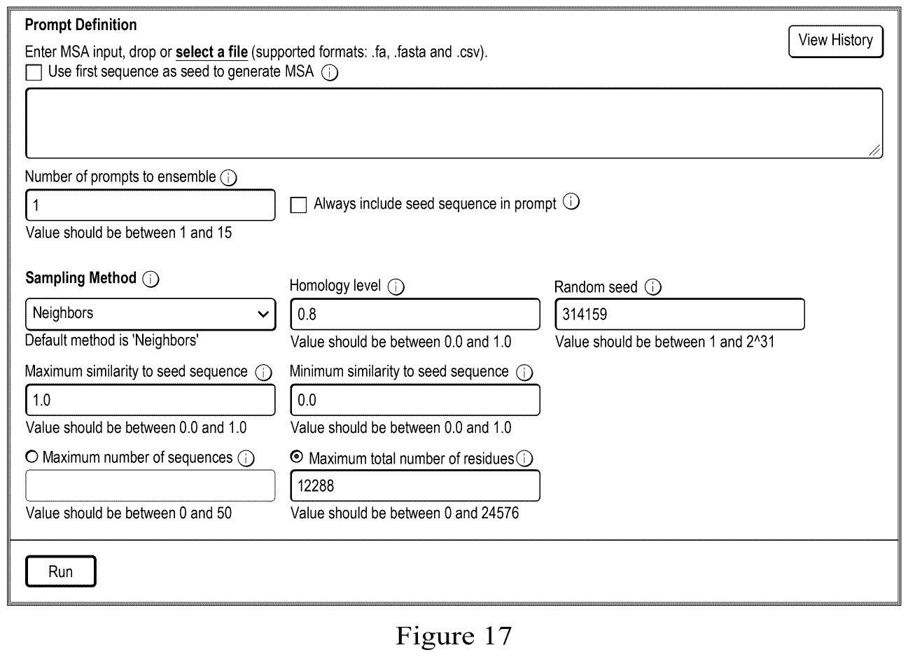

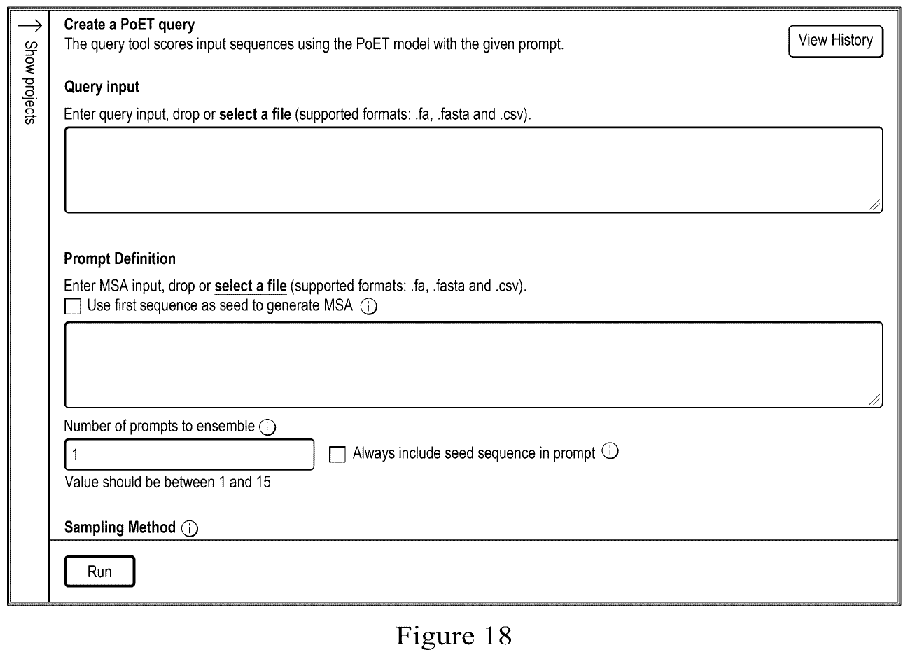

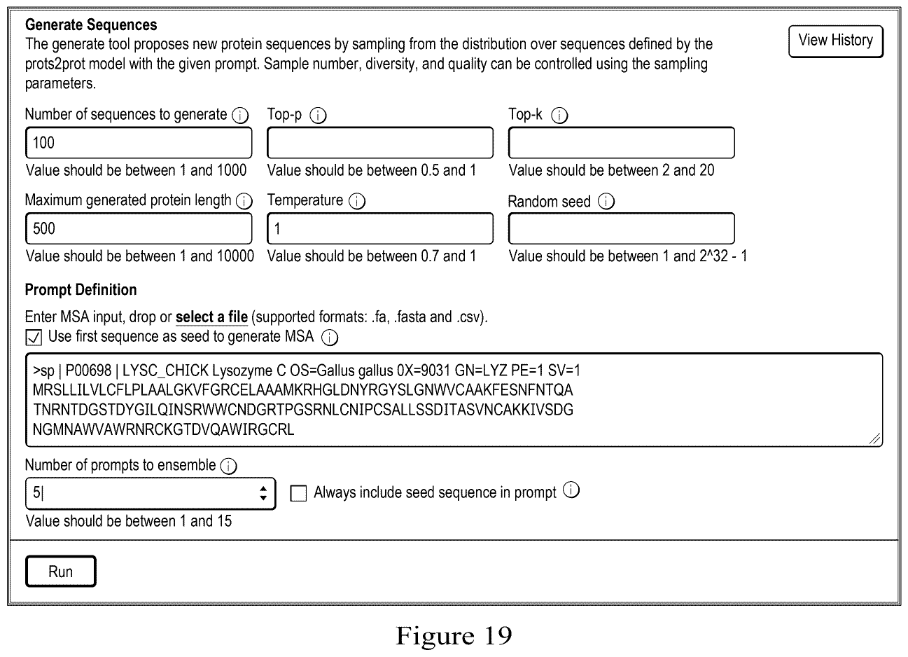

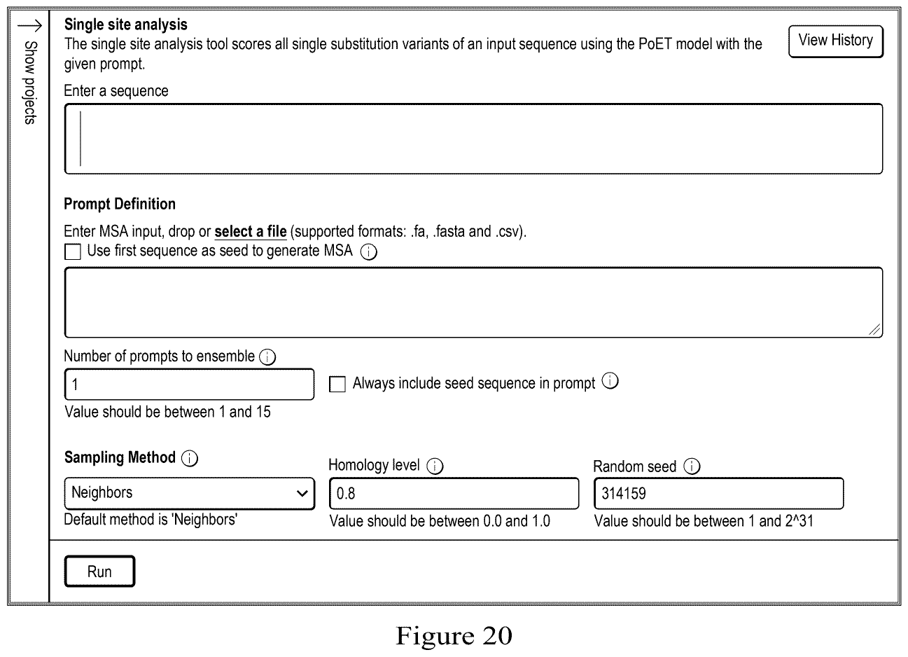

Representative interfaces for PoET prompting are depicted in FIGS. 17-20.

The nomenclature PoET is not intended to be limiting.

Other language model variants (that use the order invariant transformer layer) may be implemented. Such model variants include, for example: simplifying the second attention module in the transformer layer by removing positional encoding; in an encoder-decoder framework, decoding only one sequence at a time instead of a sequence-of-sequences (but still conditioning on a sequence-of-sequences) by replacing the NxTieredTransformerDecoderLayer in component 604 (FIG. 6) with NxTransformerDecoderLayer; by dividing up the computation by using additional encoders and decoders; by tying the weights of any two transformer layers in a model; by adding additional sequence processing layers such as RNNs or SSMs, and so forth. An example of using additional encoders and decoders may be as follows: instead of using an encoder to embed a sequence-of-sequences and decoding each sequence in the sequence-of-sequences based on the embeddings of the individual sequences, an encoder may be used to embed each sequence-of-sequences without the sequence to decode; then, embed each sequence to decode conditioned on the former using cross attention, and then use these embeddings to decode each sequence.

### Multimodal and Retrieval Augmented Generative Model of Protein Families

The following describes an alternative embodiment of the PoET model; this alternative embodiment is referred to herein as PoET-2.

PoET-2 introduces an advanced multimodal and retrieval-augmented generative model for controllable protein sequence design within a specified protein family. By leveraging homologous sequences and structures retrieved from the family of interest, PoET-2 generates novel proteins that align with the characteristics of the family. The model also allows for controllable design, enabling users to optionally constrain generated sequences to include specified sequence or structural motifs. As will be described, its encoder-decoder architecture, featuring tiered transformer layers, processes homologs in an order-equivariant manner, while its dual decoders—causal for autoregressive generation and bidirectional for contextual embeddings—offer flexibility for diverse applications, such as sequence generation and downstream predictions of structural and functional protein properties. Here, the notion of multimodal refers to sequence and structure, and the fact that both of these can be used as input to the model.

Further, PoET-2 addresses the challenge of designing novel and functional proteins that align with desired characteristics, including specified sequence and structural motifs. The model is versatile, enabling direct sequence generation, variant effect prediction through likelihood ratios, and embedding-based transfer learning for downstream tasks like protein property prediction in Bayesian optimization loops. PoET-2's multimodal and retrieval-augmented design integrates sequence and structural data while leveraging homologous proteins during inference, offering unprecedented control and precision in protein engineering.

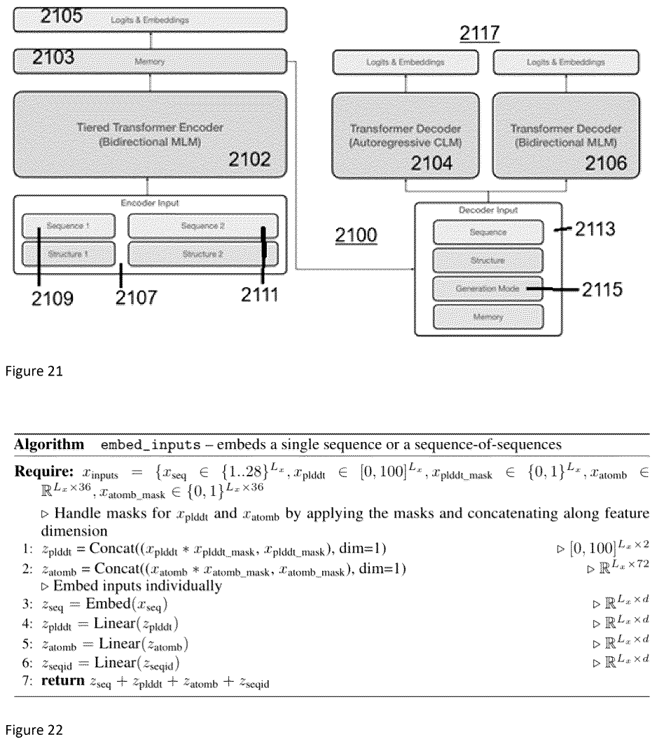

PoET-2 allows sequence to optionally be associated with a structure (sometimes referred to herein as structure “conditioning”), and it also supports new generation modes. FIG. 21 is a high level depiction of PoET-2's multimodal architecture, with the generation mode support depicted explicitly. In FIG. 21, the model architecture 2100 comprises three (3) main functional components: a hierarchical encoder 2102, an autoregressive decoder 2104, and a bidirectional decoder 2106. The encoder 2100 has associated memory 2103 for producing outputs (see the discussion above), and embeddings and logits 2105, which are the raw, unnormalized scores produced by the final layer of the model, specifically before the application of an activation function. The encoder 2102 receives an encoder input 2107, e.g., one or more sequence(s) 2109 and associated structure(s) 2111. The output(s) from the memory 2103 comprise decoder input 2113. The decoder input 2113 here includes generation mode information 2115. The decoders also have associated logits and embeddings 2117.

As will be described in more detail below, in a typical use case such as depicted, the hierarchical encoder takes a prompt as input. The prompt comprises protein sequences and optional information, e.g., a query input of sequence constraints and/or structure constraints. The hierarchical encoder produces context-rich representations. The autoregressive decoder 2104 in FIG. 21 generates full protein sequences from partial inputs (the encoded query, and a start token), while the bidirectional decoder 2106 outputs embeddings for property prediction and optimization.

The bidirectional encoder 2102 leverages a hierarchical attention mechanism that provides for in-context reasoning and generalization across the protein universe. This system processes information at two levels simultaneously; it analyzes relationships between different protein sequences in an order-independent way, while also attending to the ordered relationships between amino acids within each sequence. Unlike the first embodiment described above (PoET), this encoding is sequence-order-equivariant. Also, PoET-2 predicts both structure and sequence tokens. In addition, when structural information is available, these within-sequence relationships are further enriched by structural context, allowing PoET-2 to understand both sequence patterns and 3D arrangements. Further, and for generating outputs, PoET-2 preferably has the two decoders: the first decoder 2104 for autoregressive generation and the second decoder 2106 for bidirectional language modeling, with parameter sharing between the decoders. This dual-level processing allows PoET-2 to learn from examples that represent the protein fitness landscape of interest at inference time, whether they are evolutionary homologs, structurally or functionally similar proteins, or some other semantic grouping. Further, this architecture allows PoET-2 to generalize to small protein families and new databases without retraining.

Preferably, PoET-2 is trained on large scale natural protein sequence and structure data with a multi-task learning approach. It learns to generate diverse new protein sequences conditioned on homologous examples, to complete partially specified sequences based on a sequence and structure query, and to decode missing amino acids in a conventional masked language modeling objective given sequence-only or structure-conditioned inputs. This training regime enables two modes of operation. The autoregressive decoder 2104 specializes at generating novel proteins and completing partial sequences, while the bidirectional decoder 2106 specializes in producing context-aware embeddings that capture deep insights about protein structure and function. These embeddings serve as important features for downstream tasks, such as protein property prediction and optimization. Parameter sharing between the decoders improves parameter efficiency and improves learning across tasks.

Preferably, the autoregressive decoder 2104 is trained with a causal language modeling (CLM) objective, and the bidirectional decoder 2106 is trained with a masked language modeling (MLM) objective. Further details of this training are described below.

Preferably, PoET-2 utilizes a grammar for protein generation. In particular, and as depicted in FIG. 36, PoET-2 enables such control via its prompt 3618, which allows protein engineers to specify the desired attributes of novel generated proteins more precisely. In PoET-2, a prompt is composed of two optional parts: 1) a context 3619, and 2) a query 3621. As depicted, typically the context 3619 is a set of sequences and structures that guides PoET-2's output distribution and enables PoET-2's in-context learning and retrieval-augmented capabilities. Meanwhile, the query 3621, when utilized, encodes specific sequence and structural constraints, such as the protein's size and the presence of any signal peptides or active sites. The combination of the context and the query is sometimes referred to herein as a “flexible” or “controllable” grammar. This flexible grammar allows sequence generation to be controlled implicitly (by providing sequences/structures describing the desired distribution) and explicitly (e.g., for sequence in-filling or motif scaffolding). Prompt engineering via careful selection of the context allows PoET-2 to focus on the evolutionary, structural, or functional constraints of only the subspace of relevant proteins. For example, the prompt grammar allows PoET-2 to perform diverse sequence generation tasks from de novo sequence generation, to motif scaffolding, inverse folding, or combinations thereof. FIG. 36 also depicts the hierarchical encoder 3602, the autoregressive decoder 3604, and the bidirectional decoder 3606, together with the various inputs and outputs to these components, and representative zero-shot and supervised prediction use cases. Further details regarding these example use cases are provided below.

As noted, the PoET-2 prompt context 3619 guides PoET-2's in-context learning and retrieval-augmented generation capabilities. By prompting PoET-2 with specific sets of sequences or sequences and structures, the generative distribution can be tuned towards specific goals. For example: prompt with the sequences of protein family members for a family-specific biological sequence model; construct the prompt from homology search from a parent sequence for evolutionarily-focused variant effect predictions; build a prompt from thermophilic organisms to design heat-resistant proteins; condition on human antibody repertoire sequences to model the distribution over natural human antibodies to humanize antibodies and improve developability; use proprietary sequence or structure databases to extract insights at inference time, with no retraining required. The above are merely representative examples.

The programmable query 3621 guides protein design. In particular, the query provides a way to “programmatically” encode constraints/specifications on the generated proteins, thereby (when utilized) controlling PoET-2 outputs. It allows users to specify specific amino acids, sequence length, provide a structural template, or partial structure to accomplish specific protein generation tasks. For example, and through the query, PoET-2 can perform: free sequence generation given no query; sequence in-filling given a masked sequence query, possibly with variable length regions; inverse folding with a structure template query; motif scaffolding with a masked structure or sequence query; and others. The above are merely representative examples.

Generalizing, PoET-2 is a multimodal generative model of protein families that integrates information across two modalities, sequence and structure. As depicted back in FIG. 21, it is implemented architecturally as an encoder-decoder transformer with one encoder and two decoders. In contrast to PoET as depicted in FIG. 4, PoET-2 is multimodal because it reasons over both protein sequences and structure. The particular PoET model depicted in FIG. 4, in contrast, only reasons over sequence. Multimodality improves PoET-2's predictive capabilities, and it unlocks new generative and predictive applications that require conditioning on (or reasoning over) protein structure, such as inverse folding and motif scaffolding. PoET-2 differs structurally from PoET (including as depicted in FIG. 9) because the two decoders operative with different objectives, namely, CLM for decoder 2104, and MLM for decoder 2106. In contrast, PoET primarily addresses generative tasks with its single CLM decoder. Further, PoET-2 includes additional component(s) for processing structure inputs, and (as compared to FIG. 9) provides somewhat different outputs (as PoET-2 predicts both sequence and structure tokens).

As explained, both PoET (FIG. 4) and PoET-2 (FIG. 21) process sequences of sequences. Generally, in a sequence of sequences, one sequence is the sequence being generated, and the other sequences are being conditioned on, for example, context or query. In PoET, a single decoder processes the sequences used for conditioning, as well as the sequence being generated; the decoder is autoregressive, meaning that it is not equivariant with respect to sequence order. In PoET-2, the encoder 2102 (in FIG. 21) handles processing of the sequences used for conditioning and is bidirectional, meaning that it is equivariant to sequence order. Either decoder can be used to process the sequence being generated. Further, and building on the above, PoET-2 also leverages the flexible grammar, primarily mediated through the query, that allows users to specify explicit sequence/structure constraints on the protein they want to generate. In one example operation, the encoder encodes a prompt composed of a set of known members of a protein family (thus, only “context” and no “query”). The prompt may be the empty set, composed of no proteins, if there are no known members of the protein family. The decoders decode a new protein belonging to the protein family defined in the prompt.

Preferably, the decoders each have multiple distinct “generation modes” that allow the generated proteins to be constrained in different ways, referred to herein as: “free,” “unmask” and “target homology.” In the “free” generation mode, and as is required across all generation modes, the generated protein must belong to the protein family defined in the prompt, but it is otherwise unconstrained. In the “unmask” generation mode, which corresponds to the use of the “query” (of the flexible grammar), the generated protein must contain specific sequence or structural motifs; in this mode, the sequence and structural motifs are specified as a partially masked protein in the prompt, and the decoder is tasked with unmasking the residues at the masked positions. Masked positions can be encoded by either a “mask” token, which indicates a single residue of unknown identity, or a “gap” token, which indicates zero or more residues of unknown identity. The latter allows for the generation of regions of unknown length. The partially masked protein is referred to herein as the reference protein or sequence. In the “target homology” generation mode, the generated protein must be within a specified sequence identity range of a specific protein in the prompt. This generation mode also leverages the query. The specific protein can be any protein in the prompt, and it is also referred to herein as the reference protein or sequence. The reference may correspond to the query.

Summarizing, and via a query, the unmask and target homology generation modes allow the user to ask the PoET-2 model to respond with respect to a specific sequence in the prompt. The following are several unmask examples detailing the specific query (with the other sequences in the prompt omitted for simplicity), and the resulting decoder output of the model. To unmask a fixed number of residues (X), a partially masked query sequence MKIXXXPGARKN (with no query structure provided) generates an unmasked sequence MKIWAAPGARKN. To unmask a variable number of residues (-), a partially masked prompt sequence MKIXXXP-N (again with no query structure provided) generates an unmasked sequence MKIWAAPGARKN. For inverse folding (predicting sequence from structure), a fully masked query sequence XXXXXXXXXXXX (with query structure fully specified) generates an unmasked sequence MKIWAAPGARKN. For motif scaffolding (partial sequence and structure specification), a partially masked query sequence XXXWAAXXARXX (with query structure specified at unmasked positions) generates an unmasked sequence MKIWAAPGARKN. The following is a target homology example. For the description target homology: generate a sequence with X %-Y % sequence identity to a given sequence, and with a fully specified query sequence MKIWAAPGARKN and a sequence identity range 0-50%, the decoder output is a sequence MPTAWWPGSRKNS with 0-50% identity to the query sequence. The above sequences are merely illustrative of these operations.

Because there may be multiple sequences in the prompt, PoET-2 implements a mechanism for indicating which sequence in the prompt it should unmask or to which it should create a target homology. Preferably, this is accomplished in PoET-2 by feeding the embedding of the prompt sequence of interest as an input to the decoder. In particular, and as noted above, typically there is an embedding for every residue in a sequence. For the unmask task, an alignment is created between the prompt sequence and the sequence being decoded, and then providing (to the decoder) the embeddings of the prompt sequence based on this alignment. For the target homology task, the embedding of the first residue of the prompt sequence is provided as an input to the decoder.

The following provides additional details regarding training PoET-2.

In addition to the language modeling objectives used to train the decoders, preferably the encoder is also trained with an MLM objective. Preferably, and without being limiting, a complete loss function for the model is composed of three (3) components:

$$ℒ = {ℒ}_{MLM encoder} + {ℒ}_{CLM decoder} + {ℒ}_{MLM decoder}$$

The MLM encoder loss is a standard MLM loss. When referring to an entity that is a sequence-of-sequences, as is the case with prompts used as input to the encoder that may contain multiple proteins, the notation xⱼ⁽ⁱ⁾ is used to denote the jth residue of ith protein. Using this notation, the MLM encoder loss is:

$${ℒ}_{MLM encoder} = - {𝔼}_{x , {m}_{x}} [ \frac{1}{\vert {m}_{x} \vert} {\sum }_{i , j ∈ {m}_{x}} log p ( {x}_{j}^{( i )} \vert {x}_{\backslash {m}_{x}} ) ]$$

where mₓ is the set of masked positions in the sequence-of-sequences x.

The CLM decoder loss is a standard CLM loss additionally conditioned on (1) the encoder prompt x_{\m}_{ₓ} and (2) the generation mode g:

$${ℒ}_{CLM decoder} = - {𝔼}_{y , x , {m}_{x} , g} [ \frac{1}{{L}_{y}} {{\sum }_{i = 1}}^{{L}_{y}} log p ( {y}_{i} \vert {y}_{< i} , {x}_{\backslash {m}_{x}} , g ) ]$$

Note that y is a single sequence of length L_{y} and not a sequence-of-sequences; the notation yᵢ simply refers to the ith residue of y.

Likewise, the MLM decoder loss is a standard MLM loss additionally conditioned on x_{\m}_{ₓ} and g:

$${ℒ}_{MLM decoder} = - {𝔼}_{y , {m}_{y} , x , {m}_{x} , g} [ \frac{1}{\vert {m}_{y} \vert} {\sum }_{i ∈ {m}_{y}} log p ( {y}_{i} \vert {y}_{\backslash {m}_{y}} , {x}_{\backslash {m}_{x}} , g ) ]$$

To support unambiguous decoding of insertions indicated by gap tokens in the unmask generation mode, the target sequences in the CLM and MLM decoder loss functions, y, are adjusted per an “insertion decoding scheme” described below.

### Model Inputs

In a representative embodiment, PoET-2's sequence, structure, and generation mode inputs are represented as follows:

Sequence: protein sequences (x_{seq}) are tokenized with a single token per amino acid, a start token ($) indicating the start of a sequence, a stop token (*) indicating the end of a sequence, a mask token (X) indicating a single residue with unknown identity, and a gap token (-) indicating zero or more residues with unknown identity.

Structure: protein structures backbones (N, Cα, and C atoms) are encoded in a roto-translation invariant way using two representations: pairwise Cα distances (D) between all pairs of Cα atoms, and local structure backbone distances (x_{atomb}), namely, the 36 pairwise distances between the backbone atoms of the residue being encoded and the residues to its left and right along the sequence, i.e., 36 pairwise distances between 9 atoms. This representation is desirable because it fully specifies the protein backbone in a simple, rotation and translation invariant manner. If the atomic coordinates of an atom required for computing a distance value are masked, the distance is set to 0. To explicitly indicate that a distance value is unknown, preferably each residue is also associated with 36 Booleans ∈{0, 1} indicating whether or not this is the case. Thus, in total, and in this embodiment, each residue is associated with 72 values describing the protein.

Predicted structure confidence: the confidence in the atomic coordinates of the backbone atoms at each residue is encoded as a value ∈[0, 100] with 0 indicating low confidence and 100 indicating high confidence. In a representative embodiment, PoET-2 is trained on predicted structures in AlphaFold DB (AFDB), and it takes as input the predicted structure confidence, pLDDT, at each residue (x_{plddt}). If the input structure is predicted using a structure prediction model such as AlphaFold2, that model's predicted structure confidence (pLDDT) is used as the measure of confidence. If the input structure is experimentally solved, the confidence can be set using multiple strategies, such as setting the confidence based on the b-factor, or simply setting the confidence to 100 at all residues. If the confidence at a given position is masked, it is set to 0. In order to explicitly indicate that a confidence value is masked, each residue is also associated with a Boolean ∈{0, 1} indicating whether or not this is the case. Thus, in total, each residue is associated with 2 values describing the confidence at that residue. In

Atomic coordinates of Cα atoms: the atomic coordinates of the C_{α} atoms (x_{Cα}) are described by 3 values ∈R per residue. Additionally, a Boolean value ∈{0, 1} per residue (xₘₐₛₖ) is used to indicate positions at which the C_{α} coordinates are masked.

Generation mode query index (sometimes referred to as the reference): an optional query index (q∈N) is used as input to the decoders to indicate that the q^{th} protein in the prompt, x^{(q)}, should be used as the reference sequence for the unmask or target homology generation modes.

Target homology: the sequence identity range used for the target homology generation mode (x_{seqid}) is represented as two values ∈[0, 1] indicating the range's lower and upper bounds. The two values are repeated and shared across all residues. If the target homology generation mode is inactive (e.g., as in the encoder) or not being used in the decoder, the two values are both set to 0. In order to explicitly indicate that a sequence identity range is not being used, each residue is also associated with a Boolean ∈{0, 1} indicating whether or not this is the case. Thus, in total, preferably each residue is associated with 3 values describing the target homology generation mode at that residue.

Summarizing the above, as used herein “structure” means the protein backbone, which is specified by the coordinates of the three atoms (nitrogen, alpha carbon, and carbon) of each residue that is common to all amino acids. The protein backbone describes the overall shape of the tertiary structure, and it does not include information about the variable side chains. A protein of length L is described natively by L×3 coordinates (one coordinate for each of N, Cα, and C). The structure representation is invariant to translations and rotations of the coordinates, meaning that any translation or rotation of the coordinates results in a description of the same structure.

Thus, and to provide the structure conditioning, the structure input to PoET-2 preferably is a translation and rotation invariant representation of the protein backbone. This representation typically has two components and is constructed from the raw coordinates as follows: (1) discretized inter-residue Cα values, and (2) per-residue pairwise backbone atom distances. Regarding (1), an L×L matrix wherein element i,j contains the “value” of the discretized distance between the Cα atoms of the ith and jth residues. “Discretized” means that the distances are encoded in buckets, e.g., bucket 1 contains distances from 1-2 meters, bucket 2 contains distances from 2-3 meters, etc. The distance range of each bucket preferably is fixed, but the “value” associated with each bucket preferably is learned. Regarding (2), for each residue, this refers to pairwise distances between all backbone atoms in the residue to its left, the residue itself, and the residue to its right. This results in the 36 distances associated with each residue, so that the full protein is described by an L×36 matrix. Furthermore, preferably the structure of each residue is associated with (3) a confidence score (the pLDDT), which lets the model know the degree of uncertainty about the actual structure of each residue. Preferably, the discretized inter-residue Cα values are implemented by biasing the attention between residues in the same sequence, and the per-residue backbone atom distances and confidence scores are implemented by simply adding them as another input to the model.

### Input Embedding

The encoder and decoders share a common input embedding space that fuses the sequence (x_{seq}), local structure backbone (x_{atomb}), and pLDDT (x_{plddt}) into a single continuous latent space via summation. This input embedding, which embeds a single sequence or a sequence-of-sequences, is depicted as Algorithm 4 (embed_inputs) in FIG. 22. The other structure inputs (x_{Cα}, xₘₐₛₖ) are used in a structure-based attention bias described below, and the other generation mode (r) input is used only in the decoders.

The following provides additional details of Algorithm 4. As described above, the encoder and decoders share a common input embedding space. This space fuses representations derived from the input sequence (x_{seq}), the local structure backbone coordinates (x_{atomb}), and the pLDDT scores (x_{plddt}). Both x_{atomb} and x_{plddt} can contain entries that are masked, for instance, due to missing structural information, padding, or masking specified by a user. For each of these features, a corresponding binary mask (e.g., x_{atomb_mask}, x_{plddt_mask}) is provided, where a value of 1 indicates an observed or valid entry, and 0 indicates a masked or invalid entry. To process these potentially masked inputs, the algorithm first applies the respective binary mask to the feature data by setting the values at masked positions to zero. Subsequently, the binary mask itself is concatenated as an additional feature channel to this modified data. These augmented representations for x_{atomb} and x_{plddt} are then linearly projected into the target embedding dimension. The sequence x_{seq} is embedded directly. Finally, these three resulting embeddings are summed to form the single continuous latent representation.

### Structure-Based Attention Bias

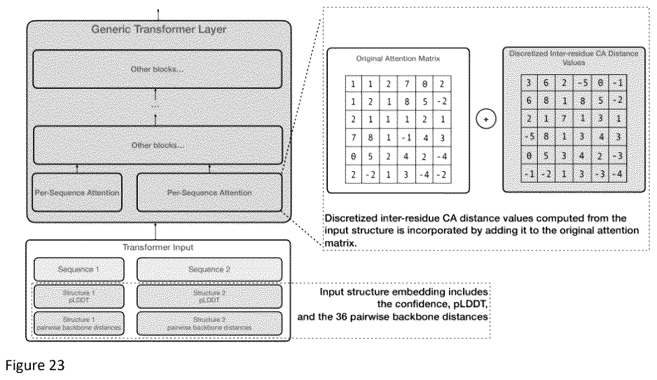

Preferably, both the encoder and the decoders use a structure-based attention bias to enhance the PoET2's ability to integrate structural information. In particular, and to enhance structural information integration, preferably PoET-2 employs a structure-based attention bias for all attention operations within individual protein sequences (but not between sequences) in both its encoder and decoders. This mechanism modifies attention scores by adding a learned bias term corresponding to the discretized pairwise Cα-Cα distance bin (D) for each residue pair. As visualized in FIG. 23, and when performing attention within an individual sequence, the attention score is modified by adding a structure-based bias term for every pair of residues. In this specific example, the bias for each pair of residues is a learned value that is associated with one of 128 bins of discretized inter-residue C_{α} distances; these bins include 125 bins of equal width between 2.5 Å and 48 Å, one bin for any distance beyond 48 Å, one bin for pairs involving at least one low-confidence residue (e.g., pLDDT<70), and one bin for pairs of positions where at least one residue has missing or masked C_{α} coordinates. In general, the bias is determined by 3D structural proximity.

### Encoder Architecture

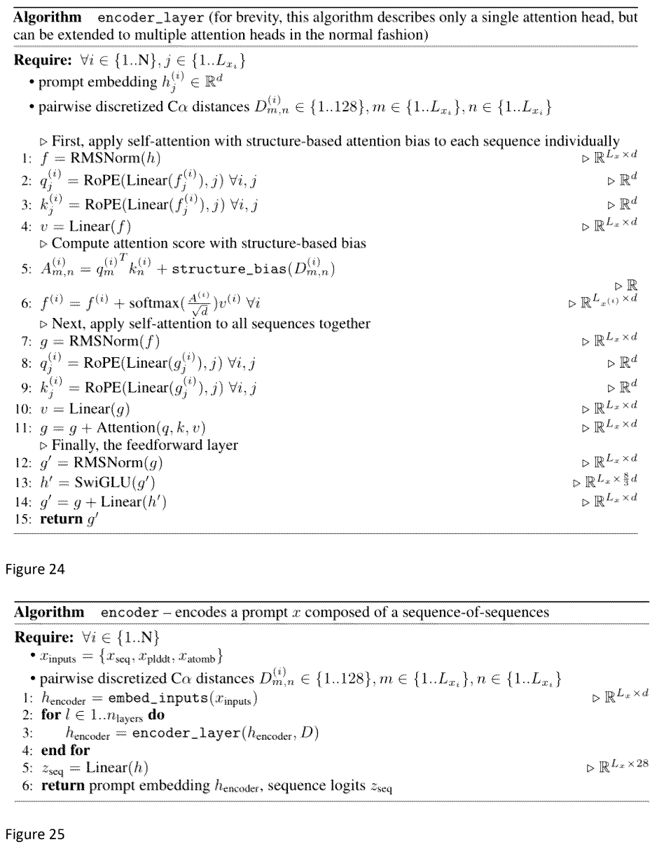

FIG. 25 is an encoder, referred to herein as Algorithm 6, which operates to encode a prompt x composed of a sequence-of-sequences. In operation, and after transforming the raw inputs xᵢₙₚᵤₜₛ into embeddings, the encoder further transforms the embeddings by applying n_{layers} of protein order equivariant encoder layers. The protein order equivariant layers are Algorithm 5, shown in FIG. 24. The encoder (Algorithm 6) adopts an architecture of standard transformer encoder layers with additional aspects (namely, rotary positional encodings, SwiGLU over MLP, and RMSNorm over LayerNorm), and other modifications to (1) ensure protein order equivariance, and (2) to improve handling of structural inputs. To this end, and to process sets of input proteins in an order equivariant way, the approach here leverages the two stage, hierarchical attention mechanism of PoET FIG. 4, but with modification. In particular, in the first stage, attention is applied only between residues of individual input proteins, and the structure-based attention bias is used in this stage. In the second stage, attention is applied between residues of all input proteins. Additionally, relative positional encodings between residues reflect the absolute positions within each protein, rather than the absolute position in the sequence-of-sequences. Attention in PoET-2's encoder is fully bidirectional, enabling the entire encoder to be protein order equivariant. The notion of being “protein order equivariant” means that a machine learning model's output predictably changes if the input protein's data representation (specifically, the sequence order) is changed in the same way. Stated another way, equivariance means that applying a transformation (like reordering an input list of amino acids) to the input data before feeding it into the model, the model's output will undergo the exact same transformation. (In the PoET model of FIG. 4, the decoder-only, autoregressive architecture permits equivariance only in each individual decoder layer, and not the entire decoder). Also, as noted above preferably the PoET-2 encoder is trained with the MLM objective rather than the CLM objective used in the PoET encoder (FIG. 4).

The encoder has two outputs, per residue embeddings of the prompt, h_{encoder}, that are used in the decoders, and per reside sequence logits, z_{seq}, that are used to compute the encoder MLM loss L_{MLM encoder}.

### Decoder Architecture

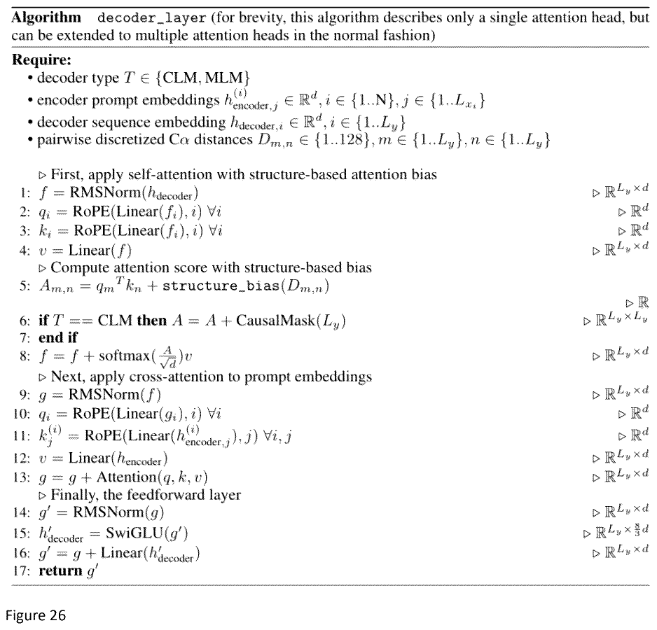

PoET-2's decoder layers are described in Algorithm 7, shown in FIG. 26. PoET-2's decoder layers adopt the architecture of a transformer decoder layer with certain modifications. In particular, modifications to the attention operations are as follows: in the first, self-attention stage, structure-based attention bias is used; in the second, cross-attention stage, protein order equivariance is maintained by using the same relative position scheme as in the second attention stage of the encoder. Additionally, when the prompt includes a query, the input embedding of the decoder is modified to encode which protein in the prompt should be used as the query. Typically, the modified input embedding of each residue is simply the average of the unmodified input embedding, and the embedding, produced by the encoder, of the corresponding residue of the query in the prompt. The decoders each decode a single sequence y, conditioned on (1) the prompt embedding h_{encoder}, and (2) an optional query index q indicating which sequence in the prompt to use as the query, if any.

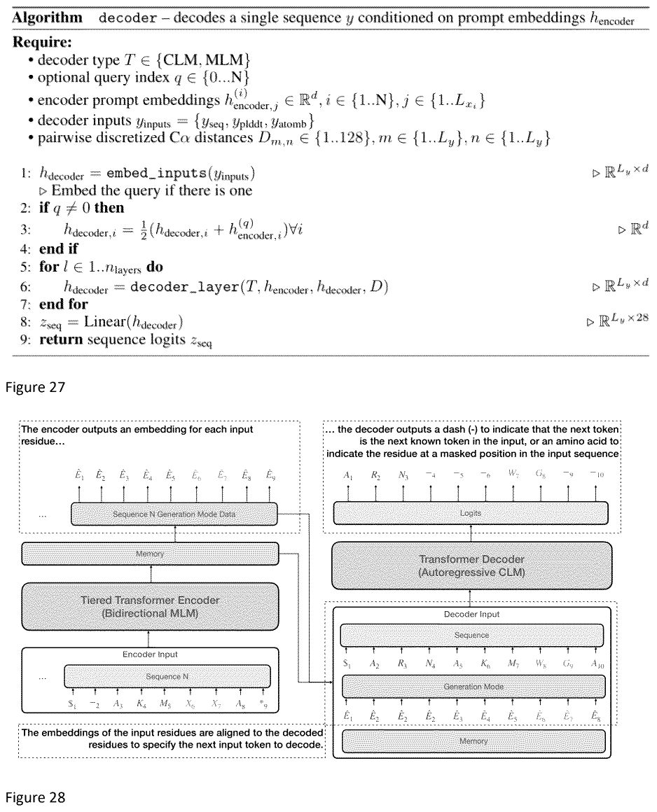

The transformations performed in each decoder are detailed in Algorithm 8, depicted in FIG. 27. The CLM decoder and MLM decoder differ in that the former uses a causal mask in the attention operations, and the latter does not. First, the input y is embedded. Next, if a query is present, the embedding of y and the corresponding embedding(s) from the query (produced by the encoder) are averaged. Then, n_{layers} of decoder layers (Algorithm 7) that are equivariant to the protein order of the prompt are applied. The decoders each have a single output, z_{seq}, that is used to compute the corresponding loss (L_{CLM decoder} for the CLM decoder and L_{MLM decode}r for the MLM decoder).

In a representative, non-limiting implementation, the CLM and MLM decoders are based on standard Seq2Seq Transformer++ decoders that decode a sequence conditioned on another sequence; as noted above, they differ from one another in that the CLM decoder uses causal attention and the MLM decoder uses bidirectional attention to support their respective use cases. In this embodiment, information about the generation mode is embedded before applying the transformer decoder layers. And, as previously mentioned, a structure-based attention bias is incorporated in the within-sequence attention phase. Also, preferably rotary positional embeddings are used between the encoder prompt embeddings and decoder sequence embeddings in the cross-attention phase. Similarly to the tiered transformer encoder layer, the positional encoding for the tokens in the sequence-of-sequences encoded by the prompt is based on each token's position in its own sequence rather than the token's position in the sequence-of-sequences.

As noted above, the decoders each have multiple distinct “generation modes” that allow the generated proteins to be constrained in different ways, referred to herein as: “free,” “unmask” and “target homology.” The following provides additional details regarding these modes.

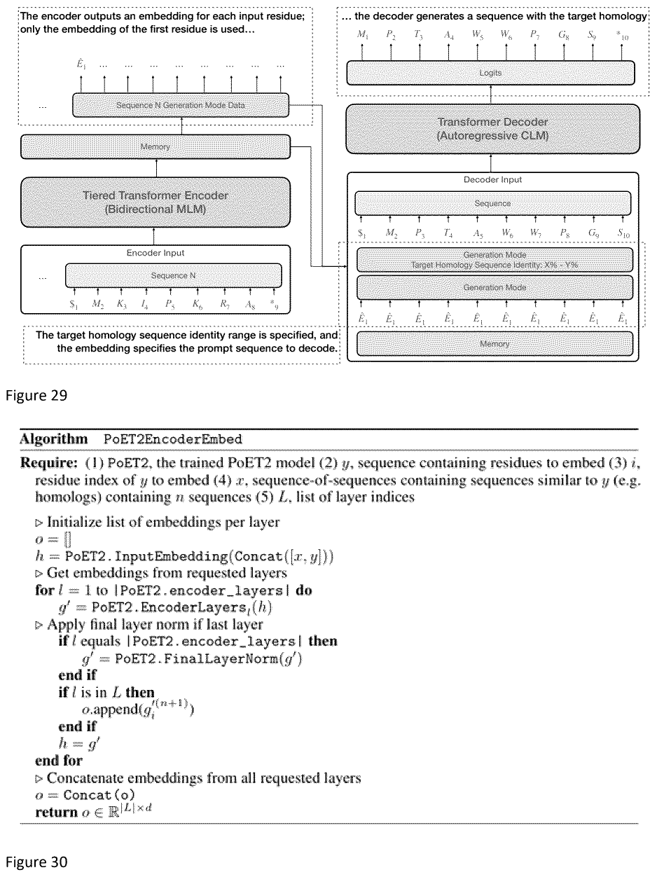

Input embedding for generation modes: As previously described, after embedding the inputs, information about the generation mode, if any, is included by averaging the embedding with the corresponding embedding of the query. In the “free” generation mode, this step is skipped (i.e. no averaging is performed), as in this mode there is no query and there are no additional constraints on the sequence being decoded. In the “unmask” generation mode, preferably each embedding of the sequence being decoded is averaged with the embedding of the corresponding query sequence embedding. A workflow for unmask generation mode is visualized in FIG. 28. To determine the correspondence between positions in the sequence being decoded and the reference sequence, an alignment between the two sequences is created to account for gap tokens in the reference sequence, which may not correspond to exactly one token in the sequence being decoded; all other tokens have a 1-to-1 mapping. When decoding a sequence defined by a user, the alignment is performed by the user and provided to the model. When decoding a sequence that is generated by the model itself, an “insertion decoding scheme” as described below preferably is used. In the “target homology” generation mode, each embedding of the decoder sequence is averaged with the embedding of the first residue of the query sequence. A workflow for target homology generation mode is depicted in FIG. 29.

The following provides additional details regarding conditioning on target homology. As noted above, PoET-2 is also trained to generate sequences that must be within a specified sequence identity range of a protein in the prompt, referred to as the query protein. The query can be any protein in the prompt whose sequence is completely known (i.e. contains no unknown or masked amino acids). This generation mode is called “target homology.” When using this generation mode, the structure of the generated protein does not have to contain any structural elements specified in the query protein. The target homology mode is implemented with two modifications to the architecture. The input embedding is augmented with a feature, x_{seqid}. This feature represents the sequence identity range as two values ∈[0, 1] indicating the range's lower and upper bounds, and is repeated across all residues. Thus, it has shape L×2. If the target homology generation mode is inactive (e.g. as in the encoder) or not being used in the decoder, these two sequence identity values are both set to 0. To prepare x_{seqid} for input embedding, it is processed similarly to other features like x_{plddt} and x_{atomb}: any values at positions intended to be masked are set to zero, and then a corresponding binary mask (1 for observed/valid, 0 for masked/invalid) is concatenated as a third channel to these two sequence identity values. This augmented 3-channel tensor for x_{seqid} is then linearly projected to the model's hidden dimension and subsequently summed with the embeddings of other input features. Further, the embedding of each residue of the generated sequence is summed with the embedding of the first residue of the query sequence.

Insertion decoding scheme: An insertion decoding scheme is a special decoding scheme used in the “unmask” generation mode that allows sequences being generated by the CLM decoder to be aligned to the reference sequence. In a standard causal Seq2Seq Transformer decoding scheme, the alignment between the reference sequence and the sequence being generated by the model may be ambiguous due to the presence of gap tokens (-) which represents an insertion of zero or more tokens. For example, suppose that the reference sequence contains the start token at the first position, the gap token at the second position, and the token A at the third position: $-A. In a normal decoding scheme, if the model predicts that the token following the start token is the token A, it is ambiguous if that token should be aligned with the gap token to indicate an insertion, or aligned with the token A to indicate that there are no insertions. To address this ambiguity, the decoders are trained to instead output the gap token when generating a token that is unmasked in the reference sequence. Continuing the example, if the model wants to generate the token A as part of an insertion aligned with the gap token in the reference sequence, then the model simply outputs the token A. If, however, the model wants to generate the token A and have it be aligned with the token A at the third position in the reference token, then the model outputs the gap token. In this case, the gap token in the reference sequence represents no insertions.

Although the insertion decoding scheme is primarily required for the CLM decoder, preferably the insertion decoding scheme is applied to the MLM decoder as well by adjusting the outputs tokens in a similar manner i.e. because the MLM decoder is trained to unmask the token at the current position (as opposed to next token prediction for the CLM decoder), if the token at the current position is unmasked, the MLM decoder is trained to output the gap token.

The following describes additional methods that preferably are implemented in a system that includes the PoET-2 model.

Obtaining Homology Augmented Per-Residue Embeddings from PoET-2

An embedding of a residue in a protein sequence is a real valued vector representing the unique context of that residue in its protein sequence. The corresponding embedding function is the function that transforms the residue into the embedding. Such an embedding function is useful when it places residues of similar contexts close to each other, and residues of differing contexts far away from each other (e.g. as measured by Euclidean distance). For example, two residues in two different protein sequences that both participate in the catalysis of the same chemical reaction may be considered similar, while a residue that does not participate in any catalysis may be considered dissimilar. The specific definition of the “context” of a residue is application dependent; the ideal embedding function works for any such definition of the context, or is able to adjust the embedding based on the definition of the context.

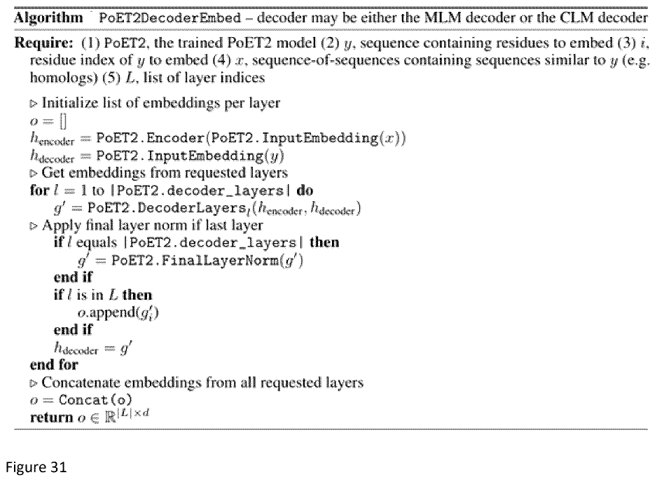

According to this aspect, PoET-2's encoder and decoders are used for creating per-residue embeddings. For the encoder, Algorithm 9 as depicted in FIG. 30 is used; for the decoder, Algorithm 10 as depicted in FIG. 31 is used. Using one of the decoders over the encoder is particularly useful when embedding residues from many sequence variants that share the same prompt, as the prompt does not have to be re-encoded for each sequence variant; this can save substantial amounts of compute. Without intending to be limiting, the MLM decoder is generally preferred over the CLM decoder as the embeddings from the MLM decoder have bidirectional context.

In a particular embodiment, PoET-2 is 182 million parameter model, structured with 12 layers and a 1024 hidden dimension in its encoder and decoders. Weights are tied between these modules to enhance parameter efficiency and promote shared representation learning. This example model is trained on 62 million sets of homologous sequences. Each set corresponds to a sequence in UniRef50 Version 2304, and contains all of its homologs in UniRef50 found using Diamond. Each sequence may optionally be associated with a predicted structure from AFDB by matching on the UniRef100 identifier. To ensure that PoET-2 sees a variety of prompts during training, and to reduce the risk of overfitting, during such training sequence and structure are randomly masked.

Summarizing, PoET-2 is a multimodal, retrieval-augmented protein language model. It learns from unaligned sequences and structure in-context and directly conditions on atomic backbone structure for protein sequence generation and representation learning. PoET-2 reasons over both sequence and structure. This enables conditioning on sequence and/or structural homologs, including structure-conditioned sequence generation from partially-observed backbone atomic structure. PoET-2 uses a context-conditioning framework featuring a hierarchical attention architecture that is fully equivariant to the order of proteins in the prompt. This eliminates the need for multi-billion parameter models, while enabling in-context learning by allowing the model to be prompted with new sequences not present in the original training data. PoET-2 is trained using both a causal language modeling objective for sequence generation and likelihood calculation, as well as a masked language modeling objective for bidirectional understanding and sequence embedding.

### Example Use Cases

A first use case is direct generation of novel proteins with user-specified functions and motifs. In particular, PoET-2's three distinct generation modes give users fine-grained control over the proteins generated by the model, allowing them to tackle a large variety of challenges in protein engineering. To use PoET-2 for sequence generation, users input a prompt containing the protein family of interest and any sequence and structure motifs that the generated sequence must contain, and the generation mode data. The prompt is processed by the encoder, and either the CLM or the MLM decoder can be used to sample the sequence. When using the CLM decoder, residues are sampled sequentially from left to right and can be sampled in combination with various techniques commonly used with other CLMs, such as top k sampling, nucleus sampling, or beam search. Likewise, the MLM decoder can be used with common techniques used with MLM decoders, such as an iterative decoding scheme.

Generation of novel proteins from a known protein family may proceed as follows. As described above, a protein family is a set of evolutionarily related proteins that share common characteristics such as structure or function. Protein engineers may want to design new proteins that belong to a known protein family because the new proteins may exhibit improved structural or functional properties, can be used as new starting sequences for further improvement, or to diversify the sequences in the protein family. PoET-2's “free” generation mode is well suited for generation of novel proteins of a known protein family. To do so, a user simply prompts PoET-2 with the known proteins from the family, and asks PoET-2 to generate new proteins without additional constraints. The proteins in the prompt may be specified by their sequence, structure, or a combination of both. The sequence and structure can be specified only partially, allowing the use of sequences and structures that may not be fully known. Compared to non-retrieval augmented models, PoET-2 has several key advantages. First, it allows PoET-2 to make use of protein data that are not in its training data without further training. In contrast, non-retrieval augmented models must be fine-tuned on the new data. This fine-tuning process can be time consuming and resource intensive, and it may sometimes fail entirely due to insufficient data. PoET-2 on the other hand, has an inductive bias for learning evolutionary constraints from only the few proteins in the prompt, which is particularly useful when the amount of data is small. Second, using PoET-2 in this manner allows the user significant flexibility in how the protein family is defined. Most commonly, a protein family is comprised of evolutionarily related proteins that are found using sequence-based homology search programs. However, sequence-based search methods may fail to find members of the protein family that have significantly diverged in sequence space. With PoET-2, users can instead use structure-based homology search programs to find additional members of the protein family, and use those proteins in the prompt. Because PoET-2 can accept any set of proteins in the prompt, the user can use any method for finding proteins to define their protein family of interest, such as a function-based method.

Generation of novel proteins with sequence and structure motifs may proceed as follows. A protein engineer may want to preserve specific sequence and structure motifs in the designed protein because the motifs may be known to be important for the protein's function. PoET-2's “unmask” generation mode allows users to specify these sequence and structure motifs. The follow paragraphs outline example use cases for specifying sequence and structure motifs that are highly prevalent in protein engineering.

Inverse folding: Inverse folding is the task of designing a protein sequence that folds into a given target structure, specified by the coordinates of the entire protein structure backbone. To use PoET-2 for this task, the user prompts PoET-2 with the context and query, namely: (1) the target protein's structure backbone fully specified, and the sequence fully unspecified and (2) any additional members of the protein family, and asks PoET-2 to “unmask” the sequence of the target protein.

Fixed enzyme active site: When improving the efficiency of an enzyme, a protein engineer may want to preserve the structure of the active site where the enzyme binds to the substrate. To use PoET-2 for this task, the user prompts PoET-2 with the context and query, namely: (1) the target protein with only the structure of the active site specified and the sequence fully unspecified and (2) any additional members of the protein family, and asks PoET2 to “unmask” the sequence of the target protein

Fixed antibody CDRs: When designing an antibody, a protein engineer may want to preserve the sequence and structure of the complementary determining regions (CDRs) of the antibody that bind to the antigen. A common reason to do this is antibody humanization, which adapts an antibody native to a non-human organism so that it can be tolerated by humans. To use PoET-2 for this task, the user prompts PoET-2 with the context and query, namely: (1) the target antibody with only the sequence and structure of the CDRs specified and (2) any additional members of the protein family (e.g. other human antibodies, or antibodies with similar CDRs), and asks PoET-2 to “unmask” the sequence of the target protein.

Miniaturization: Sometimes, a protein may already have the desired function, but is too large to be used in the target application. A protein engineer may want to miniaturize (i.e. reduce the number of residues of) this protein so that is can be used in the target application. To use PoET-2 for this task, the user prompts PoET-2 with the context and query, namely: (1) a shortened version of the original protein's sequence and/or structure in which the residues at the regions of the original protein that the user hypothesizes can be shortened are replaced with masked regions that are shorter than the length of the original regions and (2) any additional members of the protein family, and asks PoET-2 to “unmask” the sequence of the target protein.

Generation of novel proteins with a specific degree of novelty may proceed as follows. When designing a novel protein of a known protein family, a protein engineer may wish to control the degree of novelty. For example, consider a scenario where the user starts with an existing protein and wants to design a new protein that is less than 95% similar to this protein. On the other hand, the user may also want to ensure that the designed protein is at least 80% similar to this protein to improve the chance of designing a functional protein. To use PoET-2 for this task, the user prompts PoET-2 with query and context, namely: (1) the target, i.e. existing, protein and (2) additional members of the protein family, and then uses the “target homology” generation mode with the desired sequence identity range to the target protein.

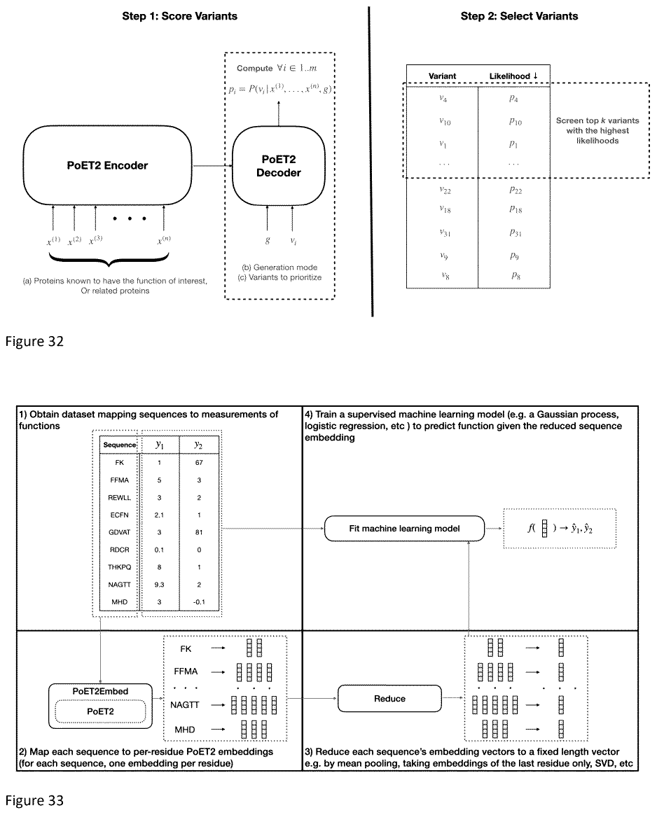

Another use case is variant prioritization. Often, there exists known proteins that perform the function of interest, but it is desired to further improve the protein, e.g. to increase its efficiency, to increase the range of temperatures for which the protein performs the function, etc. One approach to optimizing known proteins is to generate and screen many variants of those proteins, with the hope that one of these variants have higher fitness. Because the number of variants that can be screened is limited, variant prioritization methods are needed to choose the set of variants to screen. According to this aspect, PoET-2 is used to prioritize variants by ranking the variants based on their relative likelihood. The likelihood reflects the degree to which the variant satisfies the evolutionary constraints on the proteins in the prompt, and can thus be used a proxy for the overall evolutionary fitness of the protein. The likelihood can be calculated using either the CLM decoder, for which the exact likelihood is calculated, or by using the MLM encoder or MLM decoder, for which a pseudo-likelihood is calculated using known methods (e.g., ESM-1v). The variant prioritization workflow using PoET2's CLM decoder is depicted in FIG. 32. As a variant, the prompt can be combined with a generation mode to more precisely specify the constraints on the variants. For example, consider the “Fixed enzyme active site” use case, where proteins from PoET-2 that contained the structure of a given enzyme's active site are sampled. Rather than sampling from PoET-2 to directly generate proteins, one can instead use PoET-2 with the same prompt and generation mode to rank and prioritize variants of the enzyme containing the active site.

Protein property prediction is another use case. The following subsections describe how homology augmented embeddings obtained from PoET-2 are used to develop improved supervised learning models that address challenges in protein engineering.

Sequence-to-function learning: The aim of sequence to function learning is to create a mathematical model that predicts the ability of a protein to carry out its functions by learning from an existing dataset mapping protein sequences to quantitative measurements of those functions. Such predictive models can be used in black-box optimization algorithms to propose new hypotheses for protein sequences with enhanced function that can be validated in the lab. In this example, per-residue PoET-2 embeddings are used to create high quality sequence to function models by fitting machine learning models to predict measurements of function from the per-residue PoET2 embeddings of protein sequences. Specifically, a step 1 each protein sequence is mapped to a sequence of per-residue PoET-2 embeddings, one for each residue of the protein sequence. Then, at step 2 each sequence of per-residue embeddings is reduced to a fixed length vector. Examples of such reduction functions include mean pooling, taking the embedding of the last residue of the protein sequence only, and computing a singular value decomposition (SVD). Finally, at step 3, any supervised machine learning algorithm can be used to learn a function ƒ mapping the reduced embeddings to measurements of functions y\hat{ }. The workflow for this process is illustrated in FIG. 33. If the reduction in step 2 and the machine learning model learned in step 3 are differentiable with respect to their inputs, then the entire process (steps 1-3) can be learned end-to-end, meaning that the parameters of PoET-2 used to create the per-residue embeddings can be fine-tuned simultaneously via backpropagation.

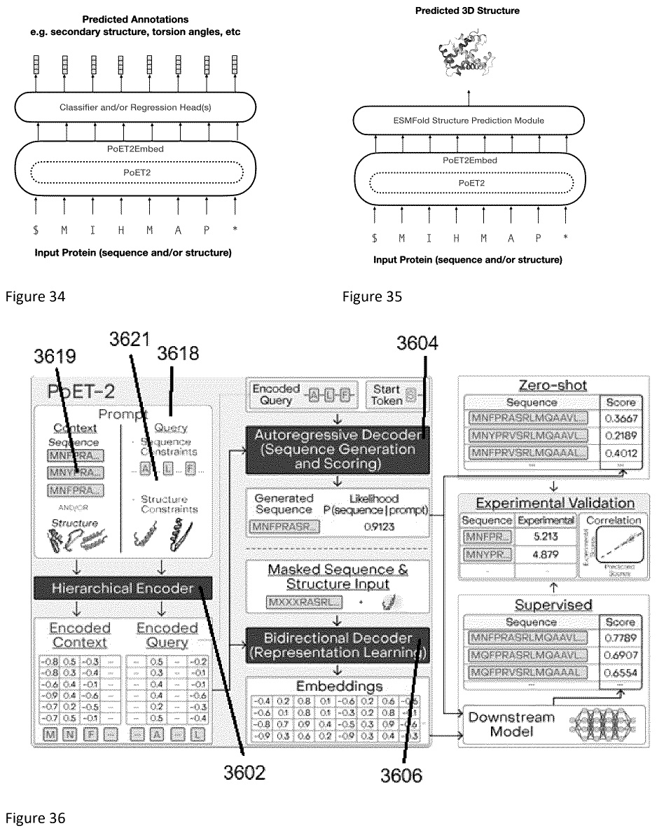

Per-residue sequence annotation: Per-residue sequence annotation is the task of annotating a set of properties for each residue in a protein sequence. Examples of such properties include secondary structure, transmembrane, torsion angles, disorder, and binding sites. PoET-2 can be applied to per-residue sequence annotation by adding classifier and/or regression head(s) on top of per-residue PoET-2 embeddings, and then finetuning the resulting model on a dataset containing annotations of the properties of interest. Any existing finetuning technique can be used. The workflow for this use case is depicted in FIG. 34. This figure depicts a per-residue sequence annotation model utilizing PoET-2.

3D structure prediction: 3D structure prediction is the task of predicting the 3D structure of a protein sequence. For this purpose, PoET-2 is applied to 3D structure prediction by replacing ESM2 per-residue embeddings with PoET-2 per-residue embeddings in an ESMFold model, and training the resulting model using the same strategy as ESMFold. FIG. 35 illustrates such a model.

Zero-shot and supervised variant effect prediction: PoET-2 encodes a set of evolutionarily relevant proteins with an equivariant encoder, and decodes proteins with either of two decoders. Log-likelihoods from the autoregressive decoder are used for zero-shot prediction, and are combined with embeddings from the bidirectional decoder for supervised prediction. This workflow is depicted in FIG. 36. The following prompt strategies are employed to facilitate this operation. First, ensemble over different prompts, where the context of each prompt contains a different subsample of sequence homologs. The ensemble prediction is simply the average of the individual predictions. Furthermore, for each context, different values for the context length (i.e. number of tokens or amino acids in the context) and maximum similarity of a sequence in the context to the Wild Type (WT) sequence are used. Utilizing PoET-2's multimodal capabilities, several methods for incorporating structure in the prompt are then considered. The first method incorporates structure in the context by associating sequences in the context with their predicted structure in AFDB, if the sequence exists in AFDB. The second method incorporates structure by adding a query to the prompt that contains the structure (but not the sequence) of WT. The use of this “inverse-folding” query instructs PoET-2 to score the likelihood that a variant sequence will fold into the same structure as WT. Although it is not necessarily desirable for a variant to adopt the same structure as WT, the inverse-folding likelihood has been shown to be predictive of protein fitness, particularly for stability related properties.

The PoET-2 model provides significant advantages. It enables structure conditioning, wherein sequences provided to the model are optionally associated with a protein structure, wherein the additional structure information enables improved modeling of the protein family. PoET-2 also allows for generation modes: “free” to generate a novel sequence from the protein family, “unmask” to generate the missing residues of a partially-specified sequence, and “target homology” to generate another sequence from the protein family that is within a specified sequence identity range of another sequence. These capabilities are also combinable to enable workflows of particular interest in protein engineering, such as inverse folding, which is the task of finding a protein sequence that folds into a specified structure. As has been described, inverse folding is done with PoET-2 by specifying the structure but not the sequence of a protein, and then asking PoET-2 to unmask the sequence.

More generally, PoET-2 provides an artificial intelligence (AI) system that reasons across modalities to understand and design proteins. By learning simultaneously from evolutionary sequence patterns and protein structure, PoET-2 extracts the fundamental principles that make proteins work. With additional in-context learning capabilities and a sophisticated, programmable design grammar, PoET-2 is able to learn from new data at runtime and solves diverse protein generation problems. This understanding allows it to navigate the vast space of possible proteins with unprecedented precision and efficiency. It also dramatically increases model efficiency. While others have focused on building ever-larger models requiring billions of parameters and massive compute resources, PoET-2 achieves superior performance with significantly fewer parameters (on the order of millions), delivering orders-of-magnitude faster inference with significantly reduced development and inference costs.

PoET-2 comprises a flexible multimodal architecture that seamlessly integrates sequence and structural information, allowing PoET-2 to operate in sequence-only or structure-guided modes, and an encoder-decoder structure that allows sequence generation and state-of-the-art representation learning. It provides a powerful context-guided learning system that discovers functional relationships at inference time, allowing PoET-2 to learn from examples without retraining, whether from public or proprietary databases. Further, it leverages a precise design grammar that enables controlled protein generation while preserving critical functional or structural elements. These architectural advances translate directly into practical capabilities that accelerate protein engineering and unlock new possibilities: superior generative modeling (protein modeling performance that would require trillion-parameter models using conventional PLM architectures—making it both more powerful and dramatically more efficient), deep structural understanding (capturing structural relationships between distant parts of proteins with unprecedented accuracy, achieving over 80% precision in contact prediction on CASP15, improving more than 10 percentage points over other models), enhanced mutation effect prediction (about how mutations affect protein function, particularly for challenging cases like insertions, deletions, and proteins with few known relatives—map sequence variants, design peptide insertions or protein truncations, or prioritize clinical mutants), and protein function mapping (carried out with orders-of-magnitude less data than existing methods—reducing experimental data needed for protein optimization by up to 30-fold, shortening iteration cycles and reducing costs to reach superior performance).

The following are additional variants or extensions. The PoET-2 model may be further trained to also predict the structure if the structure prediction head is not provided or only partially provided as an input. For the target homology generation mode, the user may also specify that the generated sequence should be within a certain sequence identity range from all sequences in the prompt, instead of just one sequence therein. The outputs generated from the model may also be conditioned on other modalities, such as text, functional annotations, secondary structure, and others.

A system that implements PoET-2 typically includes an interface through which a user configures and enters a prompt, which as noted above comprises the context, or the context and the query. Representative interfaces include a graphical user interface (GUI) on a display, a command line interface (CLI), a programmatic interface such as an Application Programming Interface (API), and the like. The PoET-2 model hyperparameters are stored in computer memory or disk, and the above-described algorithms typically are implemented as computer software.

### Enabling Technologies

Aspects of this disclosure may be practiced, typically in software, on one or more machines or computing devices. More generally, the techniques described herein are provided using a set of one or more computing-related entities (systems, machines, processes, programs, libraries, functions, or the like) that together facilitate or provide the described functionality described above. In a typical implementation, a representative machine on which the software executes comprises commodity hardware, an operating system, an application runtime environment, and a set of applications or processes and associated data, which provide the functionality of a given system or subsystem. As described, the functionality may be implemented in a standalone machine, or across a distributed set of machines. A computing device connects to the publicly-routable Internet, an intranet, a private network, or any combination thereof, depending on the desired implementation environment.

One implementation may be a machine learning-based computing platform. One or more functions of the computing platform may be implemented in a cloud-based architecture. The platform may comprise co-located hardware and software resources, or resources that are physically, logically, virtually and/or geographically distinct. Communication networks used to communicate to and from the platform services may be packet-based, non-packet based, and secure or non-secure, or some combination thereof.

Each above-described process or process step/operation preferably is implemented in computer software as a set of program instructions executable in one or more processors, as a special-purpose machine.

Representative machines on which the subject matter herein is provided may be hardware processor-based computers running an operating system and one or more applications to carry out the described functionality. One or more of the processes described above are implemented as computer programs, namely, as a set of computer instructions, for performing the functionality described. Virtual machines may also be utilized.

While the above describes a particular order of operations performed by certain embodiments of the invention, it should be understood that such order is exemplary, as alternative embodiments may perform the operations in a different order, combine certain operations, overlap certain operations, or the like. References in the specification to a given embodiment indicate that the embodiment described may include a particular feature, structure, or characteristic, but every embodiment may not necessarily include the particular feature, structure, or characteristic.

While the disclosed subject matter has been described in the context of a method or process, the subject matter also relates to apparatus for performing the operations herein. This apparatus may be a particular machine that is specially constructed for the required purposes, or it may comprise a computer otherwise selectively activated or reconfigured by a computer program stored in the computer. Such a computer program may be stored in a non-transitory computer readable storage medium, such as, but is not limited to, any type of disk including an optical disk, a CD-ROM, and a magnetic-optical disk, a read-only memory (ROM), a random access memory (RAM), a magnetic or optical card, or any type of media suitable for storing electronic instructions, and each coupled to a computer system bus.

There is no limitation on the type of computing entity that may implement a function or operation as described herein.

While given components of the system have been described separately, one of ordinary skill will appreciate that some of the functions may be combined or shared in given instructions, program sequences, code portions, and the like. Any application or functionality described herein may be implemented as native code, by providing hooks into another application, by facilitating use of the mechanism as a plug-in, by linking to the mechanism, and the like.

The functionality may be co-located or various parts/components may be separately and run as distinct functions, and in one or more locations over a distributed network.

Computing entities herein may be independent from one another, or associated with one another. Multiple computing entities may be associated with a single enterprise entity, but are separate and distinct from one another.

What is claimed here follows below.

1. A method, comprising:
providing a model of protein sequence-of-sequences configured to process both protein sequence and structure information, the model comprising an encoder, and one or more decoders, wherein each of the encoder and the one or more decoders implements a structure-based attention bias for attention operations within individual protein sequences and provides protein order equivariant processing;
receiving a prompt comprising a set of proteins; and
processing the prompt through the encoder and the one or more decoders in a protein order equivariant manner to generate a output from the model.

2. The method as described in claim 1, the structure information being a translation and rotation invariant representation of a protein backbone.

3. The method as described in claim 1, the encoder having been trained using a masked language model (MLM) objective.

4. The method as described in claim 1, wherein the one or more decoders comprise a first decoder, and a second decoder, the first decoder having been trained using a masked language model (MLM) objective, the second decoder having been trained using a causal language model (CLM) objective.

5. The method as described in claim 1, wherein the first decoder is a bidirectional decoder, and the second decoder is an autoregressive decoder.

6. The method as described in claim 1, wherein the prompt is associated with a generation mode that queries the model with respect to a specific sequence in the prompt.

7. The method as described in claim 6 wherein the generation mode is one of: a free generation mode, an unmask generation mode, and a target homology mode.

8. The method as described in claim 1, wherein the prompt also includes one of: a context, a query, and a combination thereof.

9. The method as described in claim 8, wherein the context is a protein family comprised of a set of proteins that exhibit at least one characteristic of a set of one or more given characteristics.

10. The method as described in claim 8, wherein the query is a partially specified protein that specifies, with respect to a subset of residues, an element that is one of: a sequence, a structure, and a combination thereof.

11. The method as described in claim 10, wherein responsive to receiving the prompt including the query, the output from the model is constrained to contain only proteins containing the element.

12. The method as described in claim 1, wherein the encoder and the one or more decoders share an input embedding space.

13. The method as described in claim 1, wherein the encoder comprises a bidirectional attention mechanism.

14. The method as described in claim 1, further including performing a protein engineering workflow using the output.

15. The method as described in claim 14, wherein the protein engineering workflow is one of: protein sequence design, inverse folding, motif scaffolding, miniaturization, protein generation diversification, variant prioritization, sequence-to-function prediction, per-residue annotation, structure prediction, zero-short variant effect prediction, supervised variant effect prediction.

16. The method as described in claim 8, wherein the combination of the context and the query comprise a grammar for designation of both implicit and explicit sequence and structure constraints during a protein sequence generation.

17. The method as described in claim 1, further including training the encoder of the model using a masked language model (MLM) objective.

18. A system, comprising:
a multimodal and retrieval augmented model of protein sequence-of-sequences configured to process both protein sequence and structure information, the structure information being a translation and rotation invariant representation of a protein backbone;
the model comprising an encoder, and one or more decoders, wherein each of the encoder and the one or more decoders implements a structure-based attention bias for attention operations within individual protein sequences; and
an interface that receives information that configures a prompt, the information comprising a set of proteins, and one of: a context, a query, and a combination thereof;
wherein the context is a protein family comprised of a set of proteins that exhibit at least one characteristic of a set of one or more given characteristics;
wherein the query is a partially specified protein that specifies, with respect to a subset of residues, an element that is one of: a sequence, a structure, and a combination thereof;
wherein the prompt is processed through the encoder and the one or more decoders in a protein order equivariant manner to generate a output from the model.

19. The information retrieval system as described in claim 18, wherein the encoder is a hierarchical bidirectional encoder trained using a masked language model (MLM) objective.

20. The information retrieval system as described in claim 18, wherein the information includes the combination of the context and the query.
